SEQUENCE LISTING (48 sequences)

SEQ ID NO: 1 - DNA, 26 bp, Unknown
  Adaptor
ggatcttctnnnnnnnnagaagcggg

SEQ ID NO: 2 - DNA, 11 bp, Unknown
  Adaptor
nnagaagcggg

SEQ ID NO: 3 - DNA, 10 bp, Unknown
  Adaptor
nwsswswswn

SEQ ID NO: 4 - DNA, 26 bp, Unknown
  Probe
ggatcnnnnnnnnnnnnnnnngcggg

SEQ ID NO: 5 - DNA, 52 bp, Unknown
  Probe
nnnnnnnnnnngcagcnnnggatgwswsnnnnngatgcnnnnctccagnnnn

SEQ ID NO: 6 - DNA, 26 bp, Unknown
  Adaptor
agnnnnngatgcnnnnctccagnnnn

SEQ ID NO: 7 - DNA, 26 bp, Unknown
  Adaptor
acnnnnngatgcnnnnctccagnnnn

SEQ ID NO: 8 - DNA, 26 bp, Unknown
  Adaptor
tgnnnnngatgcnnnnctccagnnnn

SEQ ID NO: 9 - DNA, 26 bp, Unknown
  Adaptor
tcnnnnngatgcnnnnctccagnnnn

SEQ ID NO: 10 - DNA, 28 bp, Unknown
  Adaptor
nnnnnnnnnnngcagcnnnggatgwsws

SEQ ID NO: 11 - DNA, 24 bp, Unknown
  Adaptor
nnnnnnnnnnngcagcaaggatcc

SEQ ID NO: 12 - DNA, 25 bp, Unknown
  Adpator
gagctcaacccatccnnnnnnnnnn

SEQ ID NO: 13 - DNA, 12 bp, Unknown
  Probe
gaaggaaggaag

SEQ ID NO: 14 - DNA, 16 bp, Unknown
  Adaptor
ggatcnnnnnnnnnnn

SEQ ID NO: 15 - DNA, 28 bp, Unknown
  Adaptor
nnnnnnnnnnnnnngcatcnnnnatgaa

SEQ ID NO: 16 - DNA, 33 bp, Unknown
  Adaptor
nnnnnnnnnnnnnngcatcnnnnatgaagatcc

SEQ ID NO: 17 - DNA, 26 bp, Unknown
  Adaptor
nnnnnnnnnnnnnnncccgcnnnnnn

SEQ ID NO: 18 - DNA, 11 bp, Unknown
  Probe
cctnnnnnagg

SEQ ID NO: 19 - DNA, 43 bp, Unknown
  Probe
nnnnnnnnnnnnnnnncctagtctaggnnnnnnnggatcnnnn

SEQ ID NO: 20 - DNA, 39 bp, Unknown
  Primer
nnnnnnnnnnnnnnnncctagnctaggnnnnnnnggatc

SEQ ID NO: 21 - DNA, 22 bp, Unknown
  Adaptor
nctaggnnnnnnnggatcnnnn

SEQ ID NO: 22 - DNA, 41 bp, Unknown
  Probe
nctaggnnnnnnnggatcnnnnnnnnnnnnggagbgagtct

SEQ ID NO: 23 - DNA, 40 bp, Unknown
  Adaptor
nctaggnnnnnnnggatcnnnnnnnnnnnnggagbgagtc

SEQ ID NO: 24 - DNA, 21 bp, Unknown
  Probe
ctaggnnnnnnnggatcnnnn

SEQ ID NO: 25 - DNA, 52 bp, Unknown
  Adaptor
nnnnnnngcagcnnnnnnnntgtgtgtgtgtgnnnnngatgcnnnnnnnnnn

SEQ ID NO: 26 - DNA, 60 bp, Unknown
  Adaptor
nnnnnnngcagcnnnnnnnntgtggtaccgtgtgtgtgtgnnnnngatgcnnnnnnnnnn

SEQ ID NO: 27 - DNA, 60 bp, Unknown
  Adaptor
nnnnnnngcagcnnnnnnnntgtgggtacctgtgtgtgtgnnnnngatgcnnnnnnnnnn

SEQ ID NO: 28 - DNA, 60 bp, Unknown
  Adaptor
nnnnnnngcagcnnnnnnnntgtgtggtaccgtgtgtgtgnnnnngatgcnnnnnnnnnn

SEQ ID NO: 29 - DNA, 60 bp, Unknown
  Adaptor
nnnnnnngcagcnnnnnnnntgtgtgggtacctgtgtgtgnnnnngatgcnnnnnnnnnn

SEQ ID NO: 30 - DNA, 60 bp, Unknown
  Adaptor
nnnnnnngcagcnnnnnnnntgtgtgtggtaccgtgtgtgnnnnngatgcnnnnnnnnnn

SEQ ID NO: 31 - DNA, 60 bp, Unknown
  Adaptor
nnnnnnngcagcnnnnnnnntgtgtgtgggtacctgtgtgnnnnngatgcnnnnnnnnnn

SEQ ID NO: 32 - DNA, 60 bp, Unknown
  Adaptor
nnnnnnngcagcnnnnnnnntgtgtgtgtggtaccgtgtgnnnnngatgcnnnnnnnnnn

SEQ ID NO: 33 - DNA, 60 bp, Unknown
  Adaptor
nnnnnnngcagcnnnnnnnntgtgtgtgtgggtacctgtgnnnnngatgcnnnnnnnnnn

SEQ ID NO: 34 - DNA, 28 bp, Unknown
  Adaptor
tgtgtgtgnnnnngatgcnnnnnnnnnn

SEQ ID NO: 35 - DNA, 12 bp, Unknown
  Adaptor
tgtgtgtgtgtg

SEQ ID NO: 36 - DNA, 90 bp, Unknown
  Probe
nnnnnnnnnnnnnntccaacnnnnnnnnnnnnnnnnnnwsnnnnnnnnnnnnnnnnnnnnnnnnnntgtgnnnnngatgcnnnnnnnnnn

SEQ ID NO: 37 - DNA, 60 bp, Unknown
  Adaptor
ctgtagtgcagcttaccacgtgtggtaccgtgtgtgtgtgcttcagatgctagtcgtcag

SEQ ID NO: 38 - DNA, 60 bp, Unknown
  Adaptor
ctgtagtgcagcttaccacgtgtgggtacctgtgtgtgtgcttcagatgctagtcgtcag

SEQ ID NO: 39 - DNA, 60 bp, Unknown
  Adaptor
ctgtagtgcagcttaccacgtgtgtggtaccgtgtgtgtgcttcagatgctagtcgtcag

SEQ ID NO: 40 - DNA, 60 bp, Unknown
  Adaptor
ctgtagtgcagcttaccacgtgtgtgggtacctgtgtgtgcttcagatgctagtcgtcag

SEQ ID NO: 41 - DNA, 60 bp, Unknown
  Adaptor
ctgtagtgcagcttaccacgtgtgtgtggtaccgtgtgtgcttcagatgctagtcgtcag

SEQ ID NO: 42 - DNA, 60 bp, Unknown
  Adaptor
ctgtagtgcagcttaccacgtgtgtgtgggtacctgtgtgcttcagatgctagtcgtcag

SEQ ID NO: 43 - DNA, 60 bp, Unknown
  Adaptor
ctgtagtgcagcttaccacgtgtgtgtgtggtaccgtgtgcttcagatgctagtcgtcag

SEQ ID NO: 44 - DNA, 60 bp, Unknown
  Adaptor
ctgtagtgcagcttaccacgtgtgtgtgtgggtacctgtgcttcagatgctagtcgtcag

SEQ ID NO: 45 - DNA, 17 bp, Unknown
  Adaptor
nngcatcaaaagatcnn

SEQ ID NO: 46 - DNA, 12 bp, Unknown
  Adaptor
nngcatcaaaag

SEQ ID NO: 47 - DNA, 12 bp, Unknown
  Adaptor
nnngcatcaaaa

SEQ ID NO: 48 - DNA, 17 bp, Unknown
  Adaptor
nnngcatcaaaaatcnn

(19) United States
(12) Patent Application Publication
(10) Pub. No.: US 2006/0211030 A1
(43) Pub. Date: Sep. 21, 2006

(54) Methods and compositions for assay readouts on multiple analytical platforms

(76) Inventor: Sydney Brenner, Ely (GB)

Correspondence Address: STEPHEN C. MACEVICZ, 21890 RUCKER DRIVE, CUPERTINO, CA, 95014, US

(21) Appl. No.: 11/377,462

(22) Filed: Mar. 16, 2006

Related U.S. Application Data
(60) Provisional application No. 60/775,098, filed on Feb. 21, 2006.
(60) Provisional application No. 60/740,480, filed on Nov. 29, 2005.
(60) Provisional application No. 60/738,852, filed on Nov. 21, 2005.
(60) Provisional application No. 60/662,167, filed on Mar. 16, 2005.

(51) Int. Cl. C12Q 1/68; C12P 19/34

(52) U.S. Cl. 435/6; 435/91.2

(57) ABSTRACT

The invention provides methods and compositions for reading out the results of multiplex assays on various analytical platforms, such as microarrays, bead arrays, electrophoresis devices, and the like. An important feature of the invention includes methods for converting different sets of oligonucleotide tags used for labeling into oligonucleotide tags specific for a particular analytical platform. The invention further includes compositions comprising oligonucleotide tags having convenient properties for labeling and conversion, particularly ligation tags that employ ligation reaction specificity as well as sequence specificity in order to discriminate between tags.

The present application claims priority from U.S. provisional applications Ser. No. 60/775,098 filed 21 Feb. 2006, Ser. No. 60/740,480 filed 29 Nov. 2005, Ser. No. 60/738,852 filed 21 Nov. 2005, and Ser. No. 60/662,167 filed 16 Mar. 2005, each one of which is incorporated by reference in its entirety.

### FIELD OF THE INVENTION

The present invention relates to methods and compositions for analyzing populations of polynucleotides, and more particularly, to methods and compositions for conducting multiplex assays using molecular tags that may be identified on multiple readout platforms.

### BACKGROUND

Many important approaches to analyzing genetic processes and variation make use of complex mixtures of oligonucleotides as probes and/or as tools for sorting and manipulating fragments or products of genomes, e.g. Brenner et al, Proc. Natl. Acad. Sci., 97: 1665-1670 (2000); Church et al, Science, 240: 185-188 (1988); Chee et al, Science, 274: 610-614 (1996); Shoemaker et al, Nature Genetics, 14: 450-456 (1996); Hardenbol et al, Nature Biotechnology, 21: 673-678 (2003); Kennedy et al, Nature Biotechnology, 21: 1233-1237 (2003); and the like. In a subset of such approaches, oligonucleotides are used as molecular tags to sort or label other molecules involved in the analytical process. A major benefit of conducting analytical reactions with molecular tags is that the tags may be designed to optimize assay sensitivity, convenience, cost, multiplexing capability, and the like. In most approaches, an analytical reaction is followed by a readout of molecular tags on a particular platform that usually involves spatial separation of the molecular tags, for example, by mass spectrometry, electrophoresis, or hybridization to a solid phase support, such as a microarray, a set of microbeads, or the like. Presently, no molecular tagging scheme has been designed with the flexibility to take advantage of more than one readout platform. For example, tags designed to be identified by hybridization are generally unsuitable for identification by electrophoretic separation, and vice versa.

The availability of a convenient molecular tagging system that could be used with multiple readout platforms would extend the use of these useful reagents and lead to improvements in analytical assays in many fields, including scientific and biomedical research, medicine, and other industrial areas where genetic measurements are important. In particular, rare genetic resources, such as libraries of genomic fragments from case and control tissues, could be tagged once for analysis and readouts on different analytical platforms.

### SUMMARY OF THE INVENTION

The invention provides methods and compositions for labeling polynucleotides and for providing multiplex readouts from assays on polynucleotides. In one aspect, the invention provides compositions of oligonucleotide tags that have properties favorable for labeling polynucleotides and for permitting readouts on various analytical platforms, such as microarrays and DNA separation instruments, such as electrophoresis devices. In this regard, the invention provides a method of converting segmented tags, that is, oligonucleotide tags made up of nucleotide or oligonucleotide subunits, into polynucleotides each having a unique length, so that the segmented tags can be identified by analysis of the size or length of such polynucleotide, which are referred to herein as “metric tags.” As explained more fully below, a segmented tag can be viewed as a number with place values, where the position (or place) of a subunit dictates the size class (i.e. the fragment set) from which a fragment is selected during the conversion for adding to a concatenate that eventually becomes a metric tag.

In another aspect, a method includes identification of members of a population of segmented tags, wherein each segmented tag of the population comprises a sequence of subunits selected from a plurality of different nucleotides or oligonucleotides, each subunit having a position within a segmented tag. In one embodiment such method is implemented by the following steps: (a) providing for each position of the segmented tags a fragment set, such fragment sets having successively larger nucleic acid fragments such that a shortest nucleic acid fragment of a next-larger fragment set has a length that is greater than or equal to that of a longest nucleic acid fragment of a next-smaller fragment set, and wherein each nucleic acid fragment within a fragment set has a different length and each fragment within a set has a one-to-one correspondence with a different subunit; (b) concatenating for each position of each segmented tag nucleic acid fragments from the fragment set corresponding to each such position and corresponding to the subunit occupying such position to form for each segmented tag a concatenate; and (c) separating the concatenates by length to identify the corresponding segmented tags.

In one aspect of the above method, the step of concatenating is carried out by cycles of sorting segmented tags by the sequences of subunits in predetermined positions and attached defined fragments to construct length-coded tags that can be separated by size. In one form, such concatenating is accomplished by the following steps: (i) sorting said segmented tags into a plurality of groups according to the identity of a subunit at a position within said segmented tags, said segmented tags having not been sorted previously from such position; (ii) attaching to each segmented tag of each group a fragment corresponding to the subunit of such group to form concatenates; (iii) combining the concatenates; and (iv) repeating steps (i) through (iii) until the segmented tags have been sorted at each position.

In another aspect, the invention provides a composition of matter comprising a set of ligation tags that comprises a plurality of member oligonucleotides with the following properties: (i) a length in the range of from six to twelve nucleotides; (ii) a duplex stability with its tag complement equivalent to that of every other member oligonucleotide; and (iii) a first terminal nucleotide and a second terminal nucleotide selected so that whenever a member oligonucleotide forms a duplex with a tag complement of another member oligonucleotide, the first terminal nucleotide and the second nucleotide each form mismatches with respect to nucleotides of the tag complement with which they are paired.

In still another aspect, the invention includes a method of identify individual polynucleotides in a mixture using ligation tags, such method comprising the following steps: (i) attaching to each individual polynucleotide in the mixture a different ligation tag to form tag-polynucleotide conjugates; (ii) generating labeled ligation tags from the tag-polynucleotide conjugates; and (iii) identifying the labeled ligation tags on a readout platform. In one embodiment, a readout platform is a solid phase support having tag complements attached, such as a microarray. In another embodiment, further steps are employed to attach unique “metric” tags to ligation tags to permit DNA separation instruments to be used as readout platforms. In such embodiments, such further steps include: (i) attaching a metric tag to each ligation tag-polynucleotide conjugate to form a metric tag-ligation tag conjugate, such that each of said ligation tags is conjugated to a unique metric tag; and (ii) separating and detecting the metric tag-ligation conjugates with a DNA separation instrument, such as a commercially available DNA sequencer.

### BRIEF DESCRIPTION OF THE DRAWINGS

FIGS. 1A-1C illustrate a conversion of dinucleotide tags into “metric” tags for a readout by electrophoretic separation.

FIGS. 2A-2B illustrate a procedure for attaching a ligation tag segment by segment to a polynucleotide.

FIGS. 3A-3G illustrate the selection of particular fragments by common sequence elements.

FIG. 4 contains a table of sequences of exemplary reagents for converting binary tags into metric tags.

### DEFINITIONS

Terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); and the like.

“Addressable” in reference to tag complements means that the nucleotide sequence, or perhaps other physical or chemical characteristics, of an end-attached probe, such as a tag complement, can be determined from its address, i.e. a one-to-one correspondence between the sequence or other property of the end-attached probe and a spatial location on, or characteristic of, the solid phase support to which it is attached. Preferably, an address of a tag complement is a spatial location, e.g. the planar coordinates of a particular region containing copies of the end-attached probe. However, end-attached probes may be addressed in other ways too, e.g. by microparticle size, shape, color, frequency of micro-transponder, or the like, e.g. Chandler et al, PCT publication WO 97/14028.

“Amplicon” means the product of a polynucleotide amplification reaction. That is, it is a population of polynucleotides, usually double stranded, that are replicated from one or more starting sequences. The one or more starting sequences may be one or more copies of the same sequence, or it may be a mixture of different sequences. Amplicons may be produced by a variety of amplification reactions whose products are multiple replicates of one or more target nucleic acids. Generally, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. No. 4,683,195; 4,965,188; 4,683,202; 4,800,159 (PCR); Gelfand et al, U.S. Pat. No. 5,210,015 (real-time PCR with “taqman” probes); Wittwer et al, U.S. Pat. No. 6,174,670; Kacian et al, U.S. Pat. No. 5,399,491 (“NASBA”); Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Complementary or substantially complementary” refers to the hybridization or base pairing or the formation of a duplex between nucleotides or nucleic acids, such as, for instance, between the two strands of a double stranded DNA molecule or between an oligonucleotide primer and a primer binding site on a single stranded nucleic acid. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

“Duplex” means at least two oligonucleotides and/or polynucleotides that are fully or partially complementary undergo Watson-Crick type base pairing among all or most of their nucleotides so that a stable complex is formed. The terms “annealing” and “hybridization” are used interchangeably to mean the formation of a stable duplex. “Perfectly matched” in reference to a duplex means that the poly- or oligonucleotide strands making up the duplex form a double stranded structure with one another such that every nucleotide in each strand undergoes Watson-Crick basepairing with a nucleotide in the other strand. The term “duplex” comprehends the pairing of nucleoside analogs, such as deoxyinosine, nucleosides with 2-aminopurine bases, PNAs, and the like, that may be employed. A “mismatch” in a duplex between two oligonucleotides or polynucleotides means that a pair of nucleotides in the duplex fails to undergo Watson-Crick bonding.

“Genetic locus,” or “locus” in reference to a genome or target polynucleotide, means a contiguous subregion or segment of the genome or target polynucleotide. As used herein, genetic locus, or locus, may refer to the position of a nucleotide, a gene, or a portion of a gene in a genome, including mitochondrial DNA, or it may refer to any contiguous portion of genomic sequence whether or not it is within, or associated with, a gene. In one aspect, a genetic locus refers to any portion of genomic sequence, including mitochondrial DNA, from a single nucleotide to a segment of few hundred nucleotides, e.g. 100-300, in length.

“Genetic variant” means a substitution, inversion, insertion, or deletion of one or more nucleotides at genetic locus, or a translocation of DNA from one genetic locus to another genetic locus. In one aspect, genetic variant means an alternative nucleotide sequence at a genetic locus that may be present in a population of individuals and that includes nucleotide substitutions, insertions, and deletions with respect to other members of the population. In another aspect, insertions or deletions at a genetic locus comprises the addition or the absence of from I to 10 nucleotides at such locus, in comparison with the same locus in another individual of a population.

“Kit” refers to any delivery system for delivering materials or reagents for carrying out a method of the invention. In the context of reaction assays, such delivery systems include systems that allow for the storage, transport, or delivery of reaction reagents (e.g., probes, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains probes.

“Ligation” means to form a covalent bond or linkage between the termini of two or more nucleic acids, e.g. oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon of a terminal nucleotide of one oligonucleotide with 3′ carbon of another oligonucleotide. A variety of template-driven ligation reactions are described in the following references, which are incorporated by reference: Whitely et al, U.S. Pat. No. 4,883,750; Letsinger et al, U.S. Pat. No. 5,476,930; Fung et al, U.S. Pat. No. 5,593,826; Kool, U.S. Pat. No. 5,426,180; Landegren et al, U.S. Pat. No. 5,871,921; Xu and Kool, Nucleic Acids Research, 27: 875-881 (1999); Higgins et al, Methods in Enzymology, 68: 50-71 (1979); Engler et al, The Enzymes, 15: 3-29 (1982); and Namsaraev, U.S. patent publication 2004/0110213.

“Microarray” refers to a solid phase support having a planar surface, which carries an array of nucleic acids, each member of the array comprising identical copies of an oligonucleotide or polynucleotide immobilized to a spatially defined region or site, which does not overlap with those of other members of the array; that is, the regions or sites are spatially discrete. Spatially defined hybridization sites may additionally be “addressable” in that its location and the identity of its immobilized oligonucleotide are known or predetermined, for example, prior to its use. Typically, the oligonucleotides or polynucleotides are single stranded and are covalently attached to the solid phase support, usually by a 5′-end or a 3′-end. The density of non-overlapping regions containing nucleic acids in a microarray is typically greater than 100 per cm², and more preferably, greater than 1000 per cm². Microarray technology is reviewed in the following references: Schena, Editor, Microarrays: A Practical Approach (IRL Press, Oxford, 2000); Southern, Current Opin. Chem. Biol., 2: 404-410 (1998); Nature Genetics Supplement, 21: 1-60 (1999). As used herein, “random microarray” refers to a microarray whose spatially discrete regions of oligonucleotides or polynucleotides are not spatially addressed. That is, the identity of the attached oligonucleoties or polynucleotides is not discemable, at least initially, from its location. In one aspect, random microarrays are planar arrays of microbeads wherein each microbead has attached a single kind of hybridization tag complement, such as from a minimally cross-hybridizing set of oligonucleotides. Arrays of microbeads may be formed in a variety of ways, e.g. Brenner et al, Nature Biotechnology, 18: 630-634 (2000); Tulley et al, U.S. Pat. No. 6,133,043; Stuelpnagel et al, U.S. Pat. No. 6,396,995; Chee et al, U.S. Pat. No. 6,544,732; and the like. Likewise, after formation, microbeads, or oligonucleotides thereof, in a random array may be identified in a variety of ways, including by optical labels, e.g. fluorescent dye ratios or quantum dots, shape, sequence analysis, or the like.

“Nucleoside” as used herein includes the natural nucleosides, including 2′-deoxy and 2′-hydroxyl forms, e.g. as described in Kornberg and Baker, DNA Replication, 2nd Ed. (Freeman, San Francisco, 1992). “Analogs” in reference to nucleosides includes synthetic nucleosides having modified base moieties and/or modified sugar moieties, e.g. described by Scheit, Nucleotide Analogs (John Wiley, New York, 1980); Uhlman and Peyman, Chemical Reviews, 90: 543-584 (1990), or the like, with the proviso that they are capable of specific hybridization. Such analogs include synthetic nucleosides designed to enhance binding properties, reduce complexity, increase specificity, and the like. Polynucleotides comprising analogs with enhanced hybridization or nuclease resistance properties are described in Uhlman and Peyman (cited above); Crooke et al, Exp. Opin. Ther. Patents, 6: 855-870 (1996); Mesmaeker et al, Current Opinion in Structual Biology, 5: 343-355 (1995); and the like. Exemplary types of polynucleotides that are capable of enhancing duplex stability include oligonucleotide N3′→P5→ phosphoramidates (referred to herein as “amidates”), peptide nucleic acids (referred to herein as “PNAs”), oligo-2′-O-alkylribonucleotides, polynucleotides containing C-5 propynylpyrimidines, locked nucleic acids (LNAs), and like compounds. Such oligonucleotides are either available commercially or may be synthesized using methods described in the literature.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C., primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. Reaction volumes range from a few hundred nanoliters, e.g. 200 nL, to a few hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al, Anal. Biochem., 273: 221-228 (1999)(two-color real-time PCR). Usually, distinct sets of primers are employed for each sequence being amplified. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26: 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Polynucleotide” or “oligonucleotide” are used interchangeably and each mean a linear polymer of nucleotide monomers. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Such monomers and their internucleosidic linkages may be naturally occurring or may be analogs thereof, e.g. naturally occurring or non-naturally occurring analogs. Non-naturally occurring analogs may include PNAs, phosphorothioate internucleosidic linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moities, or bases at any or some positions. Polynucleotides typically range in size from a few monomeric units, e.g. 5-40, when they are usually referred to as “oligonucleotides,” to several thousand monomeric units. Whenever a polynucleotide or oligonucleotide is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA or their ribose counterparts for RNA) linked by phosphodiester linkages; however, they may also comprise non-natural nucleotide analogs, e.g. including modified bases, sugars, or intemucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process are determined by the sequence of the template polynucleotide. Usually primers are extended by a DNA polymerase. Primers usually have a length in the range of from 14 to 36 nucleotides.

“Readout” means a parameter, or parameters, which are measured and/or detected that can be converted to a number or value. In some contexts, readout may refer to an actual numerical representation of such collected or recorded data. For example, a readout of fluorescent intensity signals from a microarray is the address and fluorescence intensity of a signal being generated at each hybridization site of the microarray; thus, such a readout may be registered or stored in various ways, for example, as an image of the microarray, as a table of numbers, or the like.

“Separation profile” in reference to the separation of metric tags means a chart, graph, curve, bar graph, or other representation of signal intensity data versus a parameter related to the metric tags, such as retention time, mass, length, or the like. A separation profile may be an electropherogram, a chromatogram, an electrochromatogram, a mass spectrogram, or like graphical representation of data depending on the separation technique employed. A “peak” or a “band” or a “zone” in reference to a separation profile means a region where a separated compound is concentrated. There may be multiple separation profiles for a single assay if, for example, different metric tags have different fluorescent labels having distinct emission spectra and data is collected and recorded at multiple wavelengths. In one aspect, released metric tags are separated by differences in electrophoretic mobility to form an electropherogram wherein different metric tags correspond to distinct peaks on the electropherogram. A measure of the distinctness, or lack of overlap, of adjacent peaks in an electropherogram is “electrophoretic resolution,” which may be taken as the distance between adjacent peak maximums divided by four times the larger of the two standard deviations of the peaks. Preferably, adjacent peaks have a resolution of at least 1.0, and more preferably, at least 1.5, and most preferably, at least 2.0.

“Solid support”, “support”, and “solid phase support” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. Microarrays usually comprise at least one planar solid phase support, such as a glass microscope slide.

“Specific” or “specificity” in reference to the binding of one molecule to another molecule, such as a labeled target sequence for a probe, means the recognition, contact, and formation of a stable complex between the two molecules, together with substantially less recognition, contact, or complex formation of that molecule with other molecules. In one aspect, “specific” in reference to the binding of a first molecule to a second molecule means that to the extent the first molecule recognizes and forms a complex with another molecules in a reaction or sample, it forms the largest number of the complexes with the second molecule. Preferably, this largest number is at least fifty percent. Generally, molecules involved in a specific binding event have areas on their surfaces or in cavities giving rise to specific recognition between the molecules binding to each other. Examples of specific binding include antibody-antigen interactions, enzyme-substrate interactions, formation of duplexes or triplexes among polynucleotides and/or oligonucleotides, receptor-ligand interactions, and the like. As used herein, “contact” in reference to specificity or specific binding means two molecules are close enough that weak noncovalent chemical interactions, such as Van der Waal forces, hydrogen bonding, base-stacking interactions, ionic and hydrophobic interactions, and the like, dominate the interaction of the molecules.

As used herein, the term “Tₘ” is used in reference to the “melting temperature.” The melting temperature is the temperature at which a population of double-stranded nucleic acid molecules becomes half dissociated into single strands. Several equations for calculating the Tm of nucleic acids are well known in the art. As indicated by standard references, a simple estimate of the Tm value may be calculated by the equation. Tm=81.5+0.41 (% G+C), when a nucleic acid is in aqueous solution at 1 M NaCl (see e.g., Anderson and Young, Quantitative Filter Hybridization, in Nucleic Acid Hybridization (1985). Other references (e.g:, Allawi, H. T. & SantaLucia, J., Jr., Biochemistry 36, 10581-94 (1997)) include alternative methods of computation which take structural and environmental, as well as sequence characteristics into account for the calculation of Tm.

“Sample” means a quantity of material from a biological, environmental, medical, or patient source in which detection or measurement of target nucleic acids is sought. On the one hand it is meant to include a specimen or culture (e.g., microbiological cultures). On the other hand, it is meant to include both biological and environmental samples. A sample may include a specimen of synthetic origin. Biological samples may be animal, including human, fluid, solid (e.g., stool) or tissue, as well as liquid and solid food and feed products and ingredients such as dairy items, vegetables, meat and meat by-products, and waste. Biological samples may include materials taken from a patient including, but not limited to cultures, blood, saliva, cerebral spinal fluid, pleural fluid, milk, lymph, sputum, semen, needle aspirates, and the like. Biological samples may be obtained from all of the various families of domestic animals, as well as feral or wild animals, including, but not limited to, such animals as ungulates, bear, fish, rodents, etc. Environmental samples include environmental material such as surface matter, soil, water and industrial samples, as well as samples obtained from food and dairy processing instruments, apparatus, equipment, utensils, disposable and non-disposable items. These examples are not to be construed as limiting the sample types applicable to the present invention.

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, 1 RL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The invention provides methods and compositions for reading out the results of multiplex assays on various analytical platforms, such as microarrays, bead arrays, DNA separation instruments, such as electrophoresis devices, and the like. An important feature of the invention includes methods for converting different sets of oligonucleotide tags used for labeling into oligonucleotide tags specific for a particular analytical platform and compositions comprising oligonucleotide tags having convenient properties for labeling. Other important features of the invention are compositions comprising sets of particular oligonucleotide tags, particularly ligation tags, and associated reagents for implementing methods of the invention.

In one aspect, the invention provides methods for converting segmented tags into either other segmented tags or metric tags. In regard to the latter conversion, a segmented tag is like a number with place values, where the position (or place) of a subunit dictates the size class (i.e. the fragment set) from which a fragment is selected during the conversion for adding to a concatenate that eventually becomes a metric tag. As used herein, a “segmented tag” is an oligonucleotide tag made up of a sequence of subunits that may be either nucleotides or oligonucleotides. Preferably, segmented tags of a composition of the invention each have the same number of subunits and have only subunits of the same kind occupying a position in their sequence of subunits. That is, if one segmented tag of a set has the four following subunits at the indicated positions: a nucleotide at position one, a dinucleotide at position two, a 5-mer at position three, and a nucleotide at position four, then every segmented tag of the set will have the same structure. The structure of tags in different sets of segmented tags can vary widely. A structure that is selected for a particular labeling or readout function is a design choice depending on well known factors such as the size of tag desired, how many tags in a set required, the types of enzymatic processing steps that tags undergo, whether tags are used in a hybridization reaction, the degree of discrimination between members that is required, and the like. There is significant guidance in the literature for making such selections, as noted below. In one aspect, subunits of a segmented tag are single nucleotides, which may be selected from a set of natural or non-natural nucleotides, or may be selected from a subset of the natural nucleotides. In another aspect, segmented tags have subunits that are oligonucleotides. Preferably, such oligonucleotide subunits have lengths in the range of from 2 to 12 nucleotides each. In some embodiments, all subunits have equal lengths.

Another important aspect of the invention is the use of fragment sets for constructing metric tags based on the identities of subunits at the positions of a segmented tag. Usually, there is at least one fragment set for each position of a segmented tag, and the sizes of the fragments within each set do not overlap the sizes of fragments in other sets. This is in analogy with numbers with position-dependent values. That is, the position-dependent number, 532, is 5×10²+3×10¹+2×10⁰. Likewise, if a segmented tag is made up of three subunits of dinucleotides, AC or GT (in analogy to digits 0-9), and if the leftmost or first position corresponds to fragments of length 12 (for AC) and 24 (for GT), the second position, lengths 6 (for AC) and 10 (for GT), and the third position 2 (for AC) and 4 (for GT), then a segmented tag, (AC)(GT)(GT) converts into a metric tag of length 26 (=12+10+4). In one aspect, fragment sets for a segmented tag are selected so that they have successively larger nucleic acid fragments. That is, they are selected such that a shortest nucleic acid fragment of a next-larger fragment set has a length that is greater than or equal to that of a longest nucleic acid fragment of a next-smaller fragment set. Additionally, each nucleic acid fragment within a fragment set has a different length. Usually, each fragment within a set has a one-to-one correspondence with a different subunit; however, as noted below in embodiments where, during processing, it is desirable to have metric tags all of the same length (such as when amplifying the entire set in one reaction), the same subunit may correspond to a fragment and another fragment that is a size complement. Preferably, sizes of fragments in fragment sets are selected so that distinguishable bands or peaks are formed for each metric tag in a separation profile after separation.

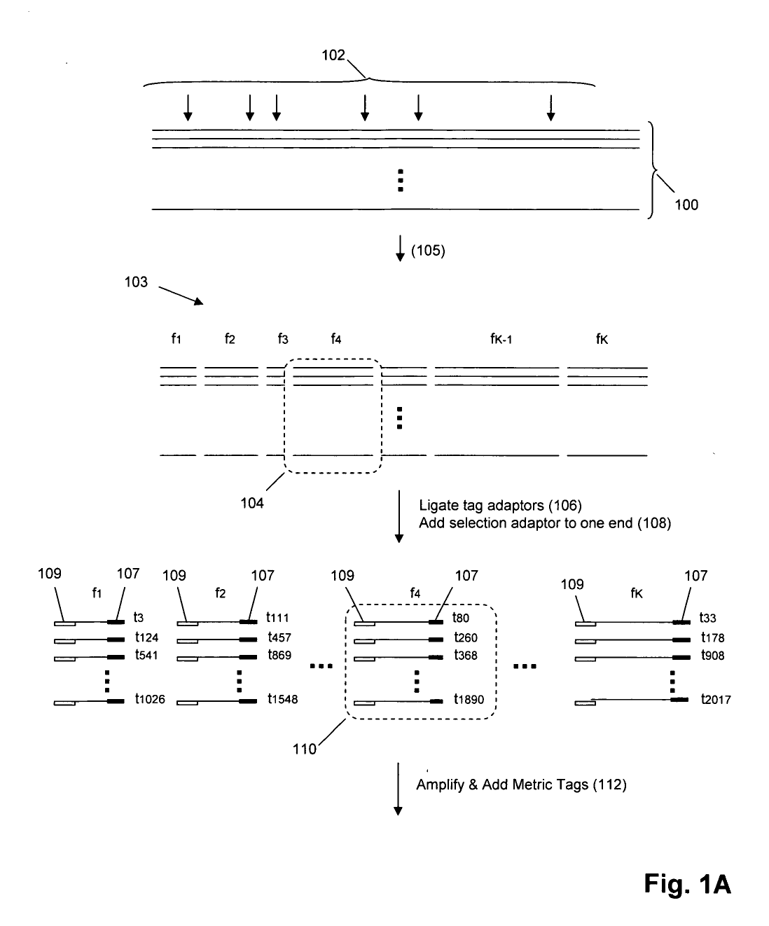

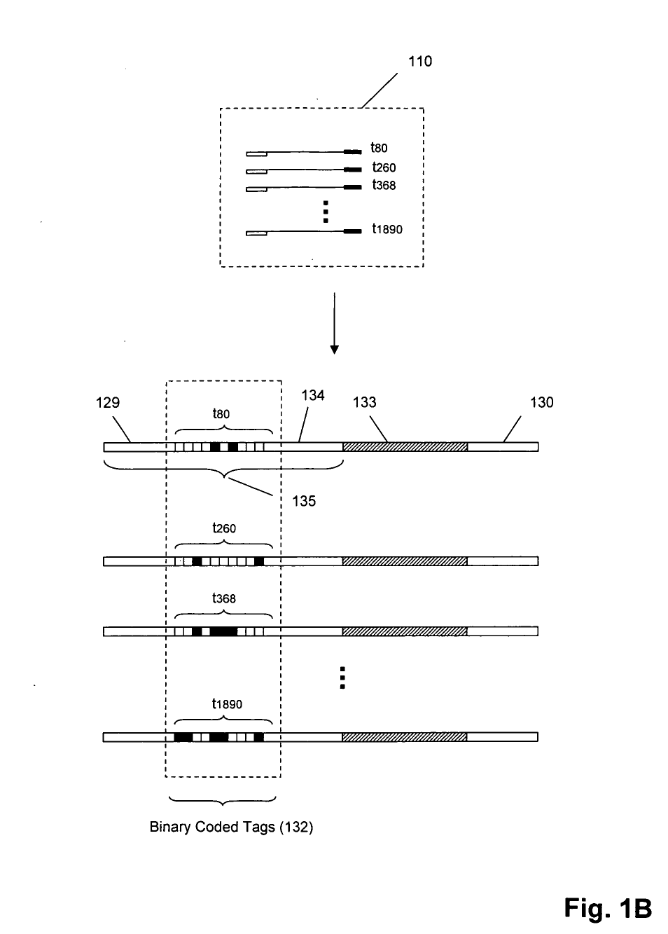

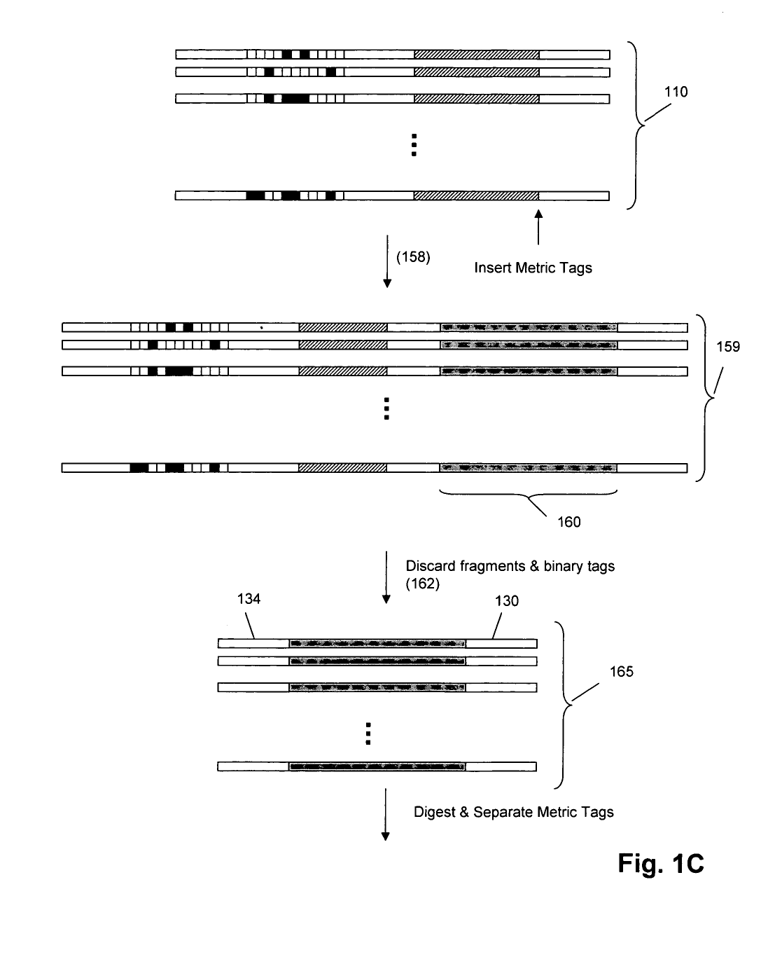

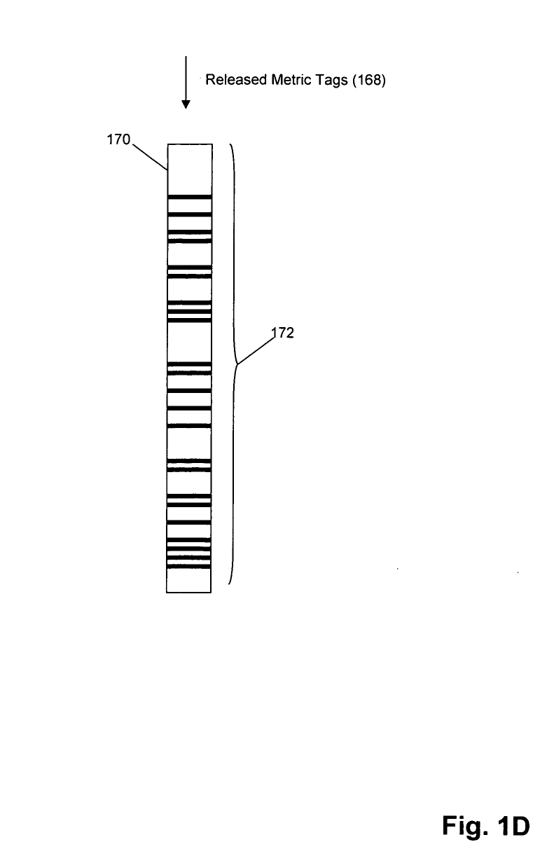

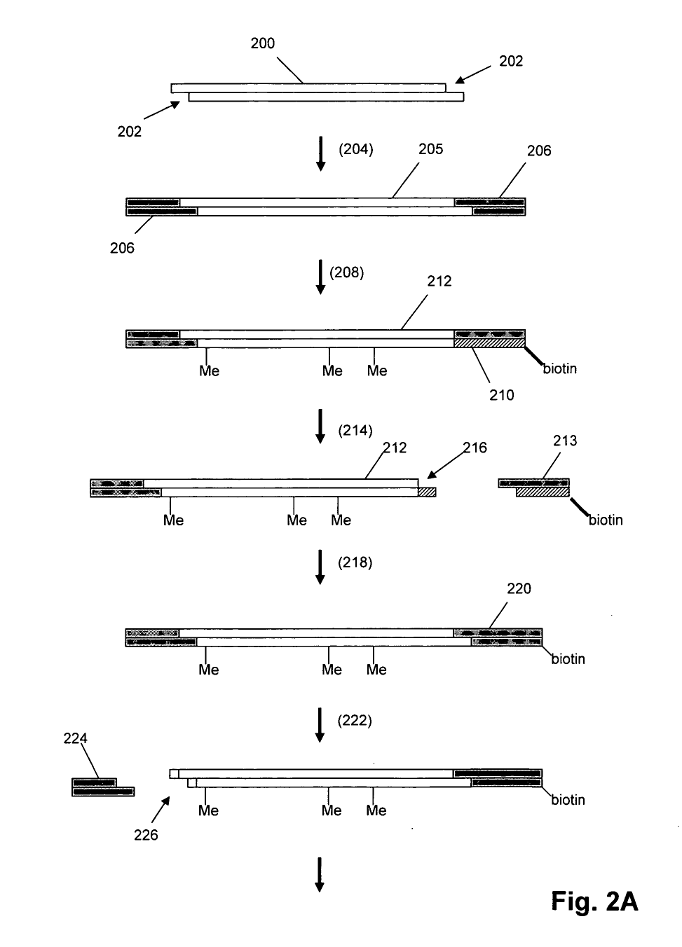

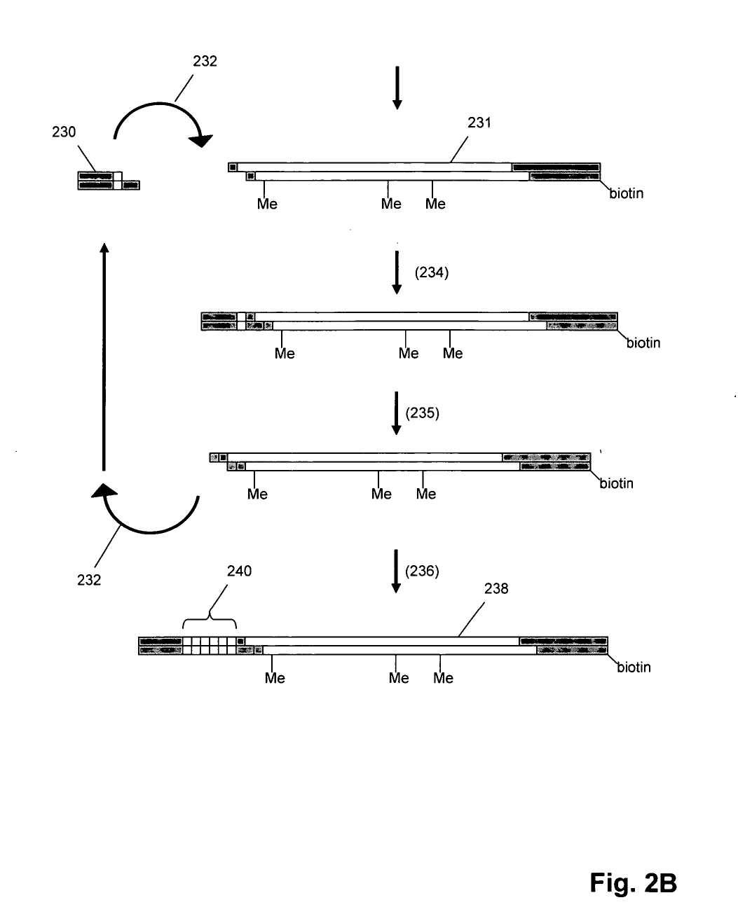

FIGS. 1A-1D provides an overview of one aspect of the invention where segmented tags, such as binary tags, are used to label genomic fragments, which after isolation by sorting by sequence are converted into metric tags for separation and enumeration. DNA (100), e.g. a sample of genomic DNA from 50 cells, extracted from s sample is digested (105) with a restriction endonuclease having recognition sites (102) so that fragments (103) are produced. Preferably, a restriction endonuclease, or a combination of restriction endonucleases, is selected that produces fragments having an expected size in the range of from 100-5000 nucleotide, and more preferably, in the range of from 200-2000 nucleotides. Other fragment size ranges are possible, however, currently available replication and amplification steps work well within the preferred ranges. The object of the method is to count the number of f₄ restriction fragments present in DNA (100) (and therefore, the sample of 50 cells). After digestion (105), adaptors (107) having complementary ends and containing oligonucleotide tags, i.e. “tag adaptors,” are ligated (106) to the fragments. If binary tags are employed (described more fully below) having 10 subunits, then 2¹⁰ or about 1024 tags are available, i.e. about 10× the number of fragments. In this example, there are about 100 fragments of each type, assuming a diploid organism. Each collection of ends of each type of fragment requires 100 tag adaptors in the ligation reaction; in effect, each collection of ends samples the population of tag adaptors. In accordance with the labeling by sampling process (see Brenner, U.S. Pat. No. 5,846,719), the tag adaptors collectively include a population of tags sufficiently large so that such a sample contains substantially all unique tags. After tag adaptors (107) are ligated, one of the tag adaptors on each fragment is exchanged for a selection adaptor (109)(which is the same for all fragments) so that each fragment has only a single tag and so that the molecular machinery necessary for carrying out sequence-specific selection is put in place. (FIG. 1B provides a more detailed illustration of the structure of the fragments at this point). One way to exchange a tag adaptor for a selection adaptor is described below and in FIGS. 2A-2B. After fragments of interest (110) have both adaptors attached, they are sorted from the rest of the fragments by the sequence-specific sorting process described in Appendix I. Briefly, such sorting is accomplished by repeated cycles of primer annealing to the selection adaptor, primer extension to add a biotinylated base only if fragments have a complement identical to that of the desired fragments, removing the biotinylated complexes, and replicating the captured fragments. That is, the selection is based on the sequence of the fragments adjacent to selection adaptor (109), which should be the same for every fragment. One controls the fragments selected by controlling which incorporated nucleotide has a capture moiety in each cycle, as described in Appendix I.

FIG. 1B illustrates a structure of fragments having different adaptors at different ends, sometimes referred to herein as “asymmetric” fragments. Exemplary fragments (110) are redrawn to show more structure. The fragments each comprise selection adaptor (129), binary tags (132), primer binding site (134), restriction fragment (133), and primer binding site (130). The binary nature of the binary tags are shown by indicating words as open and darkened boxes; that is, there are two choices of word at each position. For tag, t₈₀, the binary number for 80 is represented in the pattern of words, which, if an open box is 0 and a darkened box is 1, is simply binary 80 written in reverse order.

FIG. 1C shows fragments (110) noting the location that fragments are inserted during assembly of the metric tags in accordance with the process (158) disclosed below. After the metric tags are completely assembled, the binary tags and restriction fragment can be cleaved from fragments (159) to give metric tags (165), which may, for example, be replicated using a biotinylated primer, captured, and digested to release the single stranded metric tags to be separated using conventional techniques. (For example, the captured strands are digested with appropriate nicking and/or restriction endonucleases having recognition sites in primer binding sites (130) and (134)). After loading onto electrophoretic separation column (170), the metric tags are separated and counted to give the number of restriction fragments in the original sample.

### Attaching Tags to Polynucleotides

A method of attaching ligation tags of the invention to polynucleotides is illustrated in FIGS. 2A-2B. Polynucleotides (200) are generated that have overhanging ends (202), for example, by digesting a sample, such as genomic DNA, cDNA, or the like, with a restriction endonuclease. Preferably, a restriction endonuclease is used that leaves a four-base 5′ overhang that can be filled-in by one nucleotide to render the fragments incapable of self-ligation. For example, digestion with Bgl II followed by an extension with a DNA polymerase in the presence of dGTP produces such ends. Next, to such fragments, first-segment adaptors (206) are ligated (204). First-segment adaptors (206) (i) attach a first segment of a ligation tag to both ends of each fragment (200). First-segment adaptors (206) also contain a recognition site for a type IIs restriction endonuclease that preferably leaves a 5′ four base overhang and that is positioned so that its cleavage site corresponds to the position of the newly added segment, as described more fully in the examples below. (Such cleavage allows segments to be added one-by-one by use of a set of adaptors containing successive pairs of segments). In one aspect, a first-segment adaptor (206) is separately ligated to fragments (200) from each different individual genome.

In order to carry out enzymatic operations at only one end of adaptored fragments (205), one of the two ends of each fragment is protected by methylation and operations are carried out with enzymes sensitive to 5-methyideoxycytidine in their recognition sites. Adaptored fragments (205) are melted (208) after which primer (210) is annealed as shown and extended by a DNA polymerase in the presence of 5-methyldeoxycytidine triphosphate and the other dNTPs to give hemi-methylated polynucleotide (212). Polynucleotides (212) are then digested with a restriction endonuclease that is blocked by a methylated recognition site, e.g. Dpn 11 (which cleaves at a recognition site internal to the Bgl II site and leaves the same overhang). Accordingly, such restriction endonucleases must have a deoxycytidine in its recognition sequence and leave an overhanging end to facilitate the subsequent ligation of adaptors. Digestion leaves fragment (212) with overhang (216) at only one end and free biotinylated fragments (213). After removal (218) of biotinylated fragments (213) (for example by affinity capture with avidinated beads), adaptor (220) may be ligated to fragment (212) in order to introduce sequence elements, such as primer binding sites, for an analytical operation, such as sequencing, SNP detection, or the like. Such adaptor is conveniently biotinylated for capture onto a solid phase support so that repeated cycles of ligation, cleavage, and washing can be implemented for attaching segments of the ligation tags. After ligation of adaptor (220), a portion of first-segment adaptor (224) is cleaved so that overhang (226) is created that includes all (or substantially all) of the segment added by adaptor (206). After washing to remove fragment (224), a plurality of cycles (232) are carried out in which adaptors (230) containing pairs of segments are successively ligated (234) to fragment (231) and cleaved (235) to leave an additional segment. Such cycles are continued until the ligation tags (240) are complete, after which the tagged polynucleotides may be subjected to analysis directly, or single strands thereof may be melted from the solid phase support for analysis.

### Ligation Tags

In one aspect, methods of the invention employ oligonucleotide tags that achieve discrimination both by sequence differences and by ligation. Such tags are referred to herein as “ligation tags.” In one aspect, ends of ligation tags are correlated in that if one end matches, which is required for ligation, the other end matches as well. The sequences also allow the use of a special set of enzymes which can create overhangs of (for example) eight bases required for a set of 4096 different sequences. In one aspect, ligation tags of a set each have a length in the range of from 6 to 12 nucleotides, and more preferably, from 8 to 10 nucleotides. In one aspect, a set of ligation tags is selected so that each member of a set differs from every other member of the same set by at least one nucleotide. In the following disclosure, it is assume that a starting DNA is obtainable having the following form:

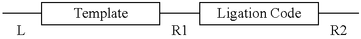

where L is a sequence to the “left” of the template that may be preselected, and R1 and R2 are primer binding sites (to the “right” of the template)In one aspect, nucleotide sequences of ligation tags in a set, i.e. ligation codes, may be defined by the following formula:

5′-Y[NN]Z[NN]Y

where Y is A, C, G, or T; N is any nucleotide; and Z is (5′→3′) GT, TG, CA, or AC. The central doublet, Z, is there so that restriction enzymes can be used to create the overhangs. Note ends of the tags are correlated, so if one does not ligate, the other will not either. Thus, the ends and the middle pair differ by 2 bases out of 8 from nearest neighbors, i.e. 25%, whereas the inners differ by one base in 8, i.e. 12.5%. Note that the above code may be expanded to give over 16,000 tags by adding an additional doublet, as in the formula: 5′-Y[NN]ZZ[NN]Y, where each Z is independently selected from the set of doublets.

In order to create an overhang of bases, a combination of a nicking enzyme and a type IIs restriction endonuclease having a cleavage site outside of its recognition site is used. Preferably, such type IIs restriction endonuclease leaves a 5′ overhang. Such enzymes are selected along with the set of doublets, Z, to exclude such sites from the ligation code. In one aspect, the following enzymes may be used with the above code: Nicking enzyme: N.Alw I (GGATCN₄↓); Restriction enzyme: Fau I (CCCGC(N₄/N₆)). Sap I (GCTCTTC(N₁/N₄)) may also be used as a restriction enzyme. In one example, these enzymes are used with the following segments:

| | |
|---|---|
| Enzyme | Sequence |

| | |
|---|---|
| N.A1w I | GGATG [TTCT] ↓ |
| Fau I | CCCGC [TTCT] ↓ |
| Sap I | GCTCTTC [T] ↓ |

A 5′ overhang can be created as follows, if a ligation code, designated as “[LIG8],” is present (SEQ ID NO: 1):

```
                  N.A1w I ↓ ↓
5′ . . . GGATCTTCT[LIG8]AGAAGCGGG . . . 3′
3′ . . . CCTAGAAGA[LIG8]TCTTCGCCC . . . 5′
                                              ↑ Fau I
```

When this structure is cleaved as shown above, two pieces are formed (SEQ ID NO: 2):

| | |
|---|---|
| 5′ . . . GGATCTTCT | pNNAGAAGCGGG . . . 3′ |
| 3′ . . . CCTAGAAGA[LIG8]p | TCTTCGCCC . . . 5′ |

where “p” represents a phosphate group.

As described above, the doublet code, Z, consisted of TG, GT, AC, and CA. These differ from each other by two mismatches and a 5 word sequence providing 1000 different sequences has a discrimination of 2 bases in 10. Another way to consider such a doublet structure is to define symbols c=C or G, a=A or T. The above code can then be expressed as ca, aa, cc, and ac. ca has the dinucleotides CA, CT, GA, and GT. Notice that in this set, each “word” differs by I mismatch from 2 members of the set but by 2 mismatches from the remaining members. The doublet code is present by definition. In fact, it is easy to see that if another repeat structure is selected, for example, caca, then many words would be found that differ by two mismatches. The c and a pairs may be arranged in any manner. For example, a sequence defining a set of 256 members could be, cacacaca, which has a clearly defined substructure, or acaaccca, which has no repeated segments. Both have 50% GC and neither has sequences that are self complementary, but the following sequence does: cacaacac.

It is well known that the melting and annealing behavior of DNA sequences depends not only on the amount GC, but more strongly on the neighboring base. Thus, cc pairs GG, CC, CG, GC contribute most to duplex stability, while ca and ac pairs make the same but lower contribution and, of the aa pairs TA is lower than the remaining three AT, AA and TT, which are like the ca and ac set. The weakness of the doublet code is that the junctions between the doublets generate cases where there are GG in one sequence and TA in another at the same place. This cannot happen with the binary code chosen above no matter how the units are arranged. Thus, cc would be uniformly high and the aa low but with the pair TA being lower than the others. Another binary system, e.g. t=G or T, s=C or A, would have a different neighbor structure in which there would be GC and TA at the same place.

It is desirable that this criterion be extended to the neighbors of the outer correlated nucleotides, which can be accomplished by requiring a sequence that begins with an a and ends with an a. A code for the inner 8 bases which satisfies these conditions is the following (SEQ ID NO: 3):

5′-Y′accacacaY”

where Y′ is G, A, T, or C, and Y“is T whenever Y′ is G, C whenever Y′ is A, G whenever Y′ is T, and A whenever Y′ is C.

In another aspect, ligation tags, or codes, can be constructed so that each sequence differs from every other in the same set by at least two bases, thereby providing greater discrimination between tags. Such tags are sets of sequences composed of the four bases A, G, C, and T, where a=A or T; and c=C or G. To preserve uniform melting and annealing behavior all “c-c” adjacencies, i.e. sequences CC, GC, GG, and CG, are forbidden. In addition, all the sequences have the same composition and, in all the cases considered below, each sequence differs from every other by at least two bases.

As a first example, five-nucleotide codes (i.e. n=5), or sequences, are considered that have a composition of a₃c₂. They can be written as follows:

aacac
acaac
caaac
acaca
caaca
cacaa

Such sequences can be considered combinations of doublets and triplets. In general, for each component one can write two sets A1 and A2. All the members of each set differ by two bases from each other, but the members of different sets differ from each other by only one base. For the doublet, aa, one can write:

| | |
|---|---|
| A1: AA | A2: TA |
| TT | AT |

The other doublets can be written in the same way:

| | |
|---|---|
| Doublet ac: | |
| B1: AC | B2: TC |
| TG | AG |
| Doublet Ca: | |
| C1: CA | C2: CT |
| GT | GA |

Likewise, triplets can be written:

| | |
|---|---|
| Triplet cac: | |
| G1: GAG | G2: GAC |
| CAC | CAG |
| GTC | GTG |
| CTG | CTC |
| Triplet aac: | |
| H1: AAG | H2: AAC |
| ATC | ATG |
| TAC | TAG |
| TTG | TTC |
| Triplet aca: | |
| I1: AGA | I2: AGT |
| TCA | TCT |
| TGT | TGA |
| ACT | ACA |
| Triplet caa: | |
| J1: GAT | J2: GAA |
| CTT | CTA |
| CAA | CAT |
| GTA | GTT |

When these are combined to provide sequences, one obtains two pairs for each 5-mer code. Thus, for example, aacac can be written as A1G1 and A2G2. Note that A1G1 differs from A2G2 in at least two bases, because A1 and A2 differ by one and G1 and G2 differ by one. The set of 5-mer sequences are written as follows:

| | | |
|---|---|---|
| aacac | A1G1 | A2G2 |
| acaac | B1H1 | 82H2 |
| acaca | B1I1 | B2I2 |
| caaac | C1H1 | C2H2 |
| caaca | C1I1 | C2I2 |
| cacaa | C1J1 | C2J2 |

Each provides two sets of 8 sequences. Thus, the total number of sequences available is 96, from which 64 are readily obtained.

Six nucleotide sequences of composition a4c₂ can also be considered:

| | | |
|---|---|---|
| aaacac | aacaca | acacaa |
| aacaac | acaaca | caacaa |
| acaaac | caaaca | cacaaa |
| caaaac | | |

These can be constructed from triplets by providing the following additional triplet to the ones listed above;

| | |
|---|---|
| Triplet aaa: | |
| K1: AAA | K2: AAT |
| TTA | TTT |
| TAT | TAA |
| ATT | ATA |

This gives the following:

| | | |
|---|---|---|
| aaacac | K1G1 | K2G2 |
| aacaac | H1H1 | H2H2 |
| acaaac | I1H1 | I2H2 |
| caaaac | J1H1 | J2H2 |
| aacaca | H1I1 | H2I2 |
| acaaca | I1I1 | I2I2 |
| caaaca | J1I1 | J2I2 |
| acacaa | I1J1 | I2J2 |
| caacaa | J1J1 | J2J2 |
| cacaaa | G1K1 | G2K2 |

Each of the pairs “X1Y1” generates 4×4=16 sequences. There are two versions of each making a total of 32 sequences. This total is 320 sequences from which 256 are chosen.

The code that can be used is a 7-mer of composition a₅c₂. Below 15 “dot” pairs are listed, 10 beginning with an “a,” and 5 with a “c.”

aca.caaa
aca.acaa
aca.aaca
aca.aaac
aaa.caca
aaa.acac
aaa.caac
aac.acaa
aac.aaca
aac.aaac
cac.aaaa
caa.caaa
caa.acaa
caa.aaca
caa.aaac

The quadruplets are composed of two sets each with 8 members, as shown below:

| caaa | | acaa | | aaca | | aaac | |
|---|---|---|---|---|---|---|---|
| M1 | M2 | N1 | N2 | O1 | O2 | P1 | P2 |
| GAAA | CAAA | AGAA | ACAA | AAGA | AACA | AAAG | AAAC |
| GATT | CATT | AGTT | ACTT | ATGT | ATCT | ATTG | ATTC |
| CATA | GATA | ACTA | AGTA | ATCA | ATGA | ATAC | ATAG |
| CAAT | GAAT | ACAT | AGAT | AACT | AAGT | AATC | AATG |
| CTAA | GTAA | TCAA | TGAA | TACA | TAGA | TAAC | TAAG |
| GTTA | CTTA | TGTA | TCTA | TTGA | TTCA | TTAG | TTAC |
| GTAT | CTAT | TOAT | TCAT | TAGT | TACT | TATG | TATG |
| CTTT | GTTT | TCTT | TGTT | TTCT | TTGT | TTTC | TTTG |
| aaaa | | caca | | caac | | acac | |
| Q1 | Q2 | S1 | S2 | T1 | T2 | V1 | V2 |
| AAAA | AAAT | GTGT | GTGA | GTTG | GTAG | TGTG | TGAG |
| ATTA | ATTT | GAGA | GACT | GAAG | GATG | AGAG | AGTG |
| ATAT | ATAA | GTCA | GTCT | GTAC | GTTC | TGAC | TGTC |
| AATT | AATA | GACT | GACA | GATC | GAAC | AGTC | ACAC |
| TAAT | TAAA | CTCT | CTCA | CTTC | CTAC | TCTC | TCAC |
| TTAA | TTAT | CACA | CACT | CAAC | CATC | ACAC | ACTC |
| TATA | TATT | CTGA | CTGT | CTAG | CTTG | TCAG | TCTG |
| TTTT | TTTA | CAGT | CAGA | CATG | CAAG | ACTG | ACAG |

Eight sequences can be selected from the 15 pairs which begin with “a” and which minimize self-complementarity. Divide into two sets:

| | |
|---|---|
| aca.caaa 5 | cac.aaac |
| aca.acaa 7 | caa.caaa |
| aca.aaca 10 | caa.acaa |
| aca.aaac 1 | caa.aaca |
| aaa.caca 6 | caa.aaac |
| aaa.acac 2 | |
| aaa.caac 3 | |
| aac.acaa 9 | |
| aac.aaca 8 | |
| aac.aaac 4 | |

In the set begining with “a” there are 10 members. All those ending in “c” will not have inverse complements; these are marked 1 to 4. 9 and 10 are self-complementary are eliminated. 8 and 7 and 6 and 5 are inverse complements but can be excluded in the final sequence.

There are 64 in each set which will be made up as follows:

| | | |
|---|---|---|
| 5 aca.caaa | I1M1 | I2M2 |
| 7 aca.acaa | I1N1 | I2N2 |
| 1 aca.aaac | I1P1 | I2P2 |
| 6 aaa.caca | K1S1 | K2S2 |
| 2 aaa.acac | K1V1 | K2V2 |
| 3 aaa.caac | K1T1 | K2T2 |
| 8 aac.aaca | H1O1 | H2O2 |
| 4 aac.aaac | H1P1 | H2P2 |

This give 512 sequences, 8 blocks of 64. These can be combined with an 8-fold sequence set, each 2 bases different from the others. This can surround the code as follows:
z-[7-base a₅c₂ code]-w
where z is selected from the group {GT, TG, CA, AC, CT, TC, GA, AG}, and w is T whenever z is GT, TG, CA, or AC, and w is A whenever z is CT, TC, GA, or AG.

Since all of the 7 base codes begin with “a,” “cc” adjacencies are excluded. Therefore, 4K sequences in 10 bases can be defined, each differing from all of the others by at least two bases. The discrimination is two out of 10, or 20%. If ligation resistance is desired at the right hand end, the sequence can be inverted to give the following:

1 caac.aca
4 caaa.aac
2 caca.aaa
3 caac.aaa
5 aaac.aca
6 acac.aaa
7 aaca.aca
8 acaa.caa

These are assembled as follows;
w-[7-base a₅c₂ code]-z
to give a final composition of a₇c₃, where w and z are defined as above.

In still another aspect, codes of 8 bases are constructed from c₃a₅ compositions from the following set of dot conjunctions:
[caaa, acaa, aaca, aaac].[caca, acac, caac] and [caca, acac, caac].[caaa, acaa, aaca, aaac]

This gives 24 pairs of which four must be eliminated as the generate a “cc.” The remaining 20 can be separated into two sets: those beginning with “a” and those ending with “a.”

1. Beginning with “a” or “ c”

| | |
|---|---|
| acaa.caca | caaa.caca |
| 1 acaa.acac | caaa.acac |
| 2 acaa.caac | caaa.caac |
| aaca.caca | caca.caaa |
| 3 aaca.acac | caca.acaa |
| 4 aaca.caac | caca.aaca |
| 5 aaac.acac | caca.aaac |
| 7 acac.acaa | caac.acaa |
| 8 acac.aaca | caac.aaca |
| 6 acac.aaac | caac.aaac |

2. Ending with “a” or “c”

| | |
|---|---|
| 7 acaa.caca | acaa.acac |
| 8 aaca.caca | acaa.caac |
| acac.acaa | aaca.acac |
| acac.aaca | aaca.acac |
| 1 caaa.caca | aaca.caac |
| 2 caca.caaa | aaac.acac |
| 3 caca.acaa | acac.aaac |
| 4 caca.aaca | caaa.acac |
| 5 caac.acaa | caaa.caac |
| 6 caac.aaca | caca.aaac |
| | caac.aaac |

As before, no inverse complements are selected if sequences beginning with “a” and ending with “C”. Similarly, chose those beginning with “c” in the second table, also the remain four are common to both tables and are the following:

acaa.caca ⊃ acac.aaca
aaca.caca ⊃ acac.acaa

which forms pairs of inverse complements. Choose one member of each set and allocate as 7 and 8 in table 1 and 2. Each dot pair gives 128 sequences, so each of these 8 sets gives I K sequences. The first set is labeled “a.” and the second “.a” and they are embedded as follows:

G[a.]T
C[a.]A
T[.a]C
A[.a]G

There is a total of 1K in each block, so this gives 4K in 10 bases, mismatches of 2, discrimination 20%, composition a₆c₄. If all 10 are used, then 5K polynucleotides can be encoded.

### Direct Readout of Ligation Tags

In one aspect, after an analytical operation is conducted in which tags are selected and labeled, such tags may be detected on an array, or microarray, of tag complements, as shown below. Selected ligation tags may be in an amplifiable segment as follows (SEQ ID NO: 4):

```
                          N.A1w I ↓ ↓
5′ [Primer L]GGATCNNNN[LIG8]NNNNGCGGG[Primer R] 3′
3′ [Primer L]CCTAGNNNN[LIG8]NNNNCGCCC[Primer R] 5′
                                                      ↑ Fau I
```

Cleavage of this structure gives the following, the upper strand of which may be labeled, e.g. with a fluorescent dye, quantum dot, hapten, or the like, using conventional techniques:

5′ [Primer L]GGATCNNNN
3′ [Primer L]CCTAGNNNN[LIG8]p-5′

This fragment may be hybridized to an array of tag complements such as the following: 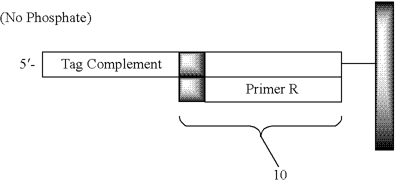
where the oligonucleotide designated as “10” may be added before or with the labeled ligation tag. 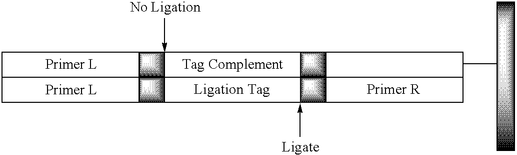
After a hybridization reaction, hybridized ligation tags are ligated to oligonucleotide “10” to ensure that a stable structure is formed. The ends between the upper Primer L and the tag complement are not ligated because of the absence of a 5′ phosphate on the tag complement. Such an arrangement permits the washing and re-use of the solid phase support. In one aspect, tag complements and the other components attached to the solid phase support are peptide nucleic acids (PNAs) to facilitate such re-use.

### Exemplary Binary Tags

In one aspect, the invention utilizes sets of dinucleotides to form unique binary tags, which can be synthesized chemically or enzymatically. In regard to chemical synthesis, large sets of tags, binary or otherwise, can be synthesized using microarray technology, e.g. Weiler et al, Anal. Biochem., 243: 218-227 (1996); Lipschutz et al, U.S. Pat. No. 6,440,677; Cleary et al, Nature Methods, 1: 241-248 (2004), which references are incorporated by reference. In one aspect, dinucleotide “words” can be assembled into a binary tag enzymatically. In one such embodiment, different adaptors are attached to different ends of each polynucleotide from each sample, thereby permitting successive cycles of cleavage and dinucleotide addition at only one end. The method further provides for successive copying and pooling of sets of polynucleotides along with the cleavage and addition steps, so that at the end of the process a single mixture is formed wherein fragments from each sample or source are uniquely labeled with an oligonucleotide tag. Identification of polynucleotides can be accomplished by recoding the oligonucleotide tags of the invention for readout on a variety of platforms, including electrophoretic separation platforms, microarrays, beads, or the like.

In one aspect, sets of binary tags for labeling multiple polynucleotides comprise a concatenation of more than one dinucleotides selected from a group, each dinucleotide of the group consisting of two different nucleotides and each dinucleotide having a sequence that differs from that of every other dinucleotide of the group by at least one nucleotide. In another aspect, none of the dinucleotides of such a group are self-complementary. In still another aspect, dinucleotides of such a group are AG, AC, TG, and TC.

Generally, dinucleotide codes for use with the invention comprise any group of dinucleotides wherein each dinucleotide of the group consists of two different nucleotides, such as AC, AG, AT, CA, CG, CT, or the like. In one aspect, dinucleotides of a group have the further property that dinucleotides of a group are not self-complementary. That is, if dinucleotides of a group are represented by the formula 5′-XY, then X and Y do not form Watson-Crick basepairs with one another. That is, preferably, XY does not include AT, TA, CG, or GC. A preferred group of dinucleotides for constructing oligonucleotide tags in accordance with the invention consists of AG, AC, TG, and TC.

The lengths of binary tags constructed from dinucleotides may vary widely depending on the number of molecules to be counted. In one aspect, when the number of molecules is in the range of from 100 to 1000, then the number of binary tags required is about 100 times the numbers in this range, or from 10⁴ to 10⁵. Thus, binary tags comprise from 14 to 17 dinucleotide subunits.

Below, reagents and methods are described for using the dinucleotide codes and resulting oligonucleotide tags of the invention. The particular selections of restriction endonucleases, oligonucleotide lengths, selection of sequences, and particular applications are provided as examples. Selections of alternative embodiments using different restriction endonucleases and other functionally equivalent enzymes, oligonucleotide lengths, and particular sequences are design choices within the purview of the invention.

### Reagents for Attaching Dinucleotides to Polynucleotides

In one aspect, the invention employs the following set of four dinucleotides: AG, AC, TG, and TC, allowing genomes to be tagged in groups of four. These are attached to ends of polynucleotides that are restriction fragments generated by digesting target DNAs, such as human genomes, with a restriction endonuclease. Prior to attachment, the restriction fragments are provided with adaptors that permit repeated cycles of dinucleotide attachment to only one of the two ends of each fragment. This is accomplished by selectively protecting the restriction fragments and adaptors from digestion in the dinucleotide attachment process by incorporating 5-methylcytosines into one strand of each of the fragment and/or adaptors. In this example, Sfa NI, which cannot cleave when its recognition site is methylated and which leaves a 4-base overhang, is employed in the adaptors for attaching dinucleotides. A similar enzyme that left a 2-base overhang could also be used, the set of reagents illustrated below being suitably modified.

Reagents for attaching dinucleotides are produced by first synthesizing the following set of two-dinucleotide structures (SEQ ID NO: 5):

```
                        LH

[symbol]

            Bbv I Bst F51
5′-N11GCAGCNNNGGATG(WS)ᵢ(WS)ⱼNNNNNGATGCNNNNCTCCAGNNNN
      N11CGTCGNNNCCTAC(WS)ᵢ(WS)ⱼNNNNNCTACGNNNNGAGGTCNNNN-5′
                                                                      Sfa NI Bpm I

[symbol]

                                                                                  RH
```

where N is A, C, G, or T, or the complement thereof, (WS)ᵢ and (WS)ⱼ are dinucleotides, and the underlined segments are recognition sites of the indicated restriction endonucleases. “LH” and “RH” refer to the left hand side and right hand side of the reagent, respectively. In this embodiment, sixteen structures containing the following sixteen different pairs of dinucleotides are produced:

| | | | |
|---|---|---|---|
| AGAG | ACAG | TGAG | TCAG |
| AGAC | ACAC | TGAC | TCAC |
| AGTG | ACTG | TGTG | TGTG |
| AGTC | ACTC | TGTC | TCTC |

Four mixtures of the above structures are created whose dinucleotide pairs can be represented as follows:

[WS]AG
[WS]AC
[WS]TG
[WS]TC

where [WS] is AG, AC, TG, or TC. Two PCRs are carried out on each of the sixteen structures, one with the left hand primer biotinylated, L, and one with the right hand primer biotinylated, R. Pool L amplicons to form the mixtures above, digest L amplicons with BstF51, and remove the LH end as well as any uncut sequences or unused primers to give mixtures containing the following structures (SEQ ID NO: 6, 7, 8, and 9):

| | |
|---|---|
| AGNNNNNGATGCNNNNCTCCAGNNNN | (I) |
| (WS) TCNNNNNCTACGNNNNGAGGTCNNNN | |
| ACNNNNNGATGCNNNNCTCCAGNNNN | (II) |
| (WS) TGNNNNNCTACGNNNNGAGGTCNNNN | |
| TGNNNNNGATGCNNNNCTCCAGNNNN | (III) |
| (WS) ACNNNNNCTACGNNNNCAGGTCNNNN | |
| TCNNNNNGATGCNNNNCTCCAGNNNN | (IV) |
| (WS) AGNNNNNCTACGNNNNGAGGTCNNNN | |

where WS is AG, AC, TG, or TC. For R amplicons, after PCR, pool all, cut with Bpm 1, and remove the right hand end to give a mixture of the following structures (SEQ ID NO: 10):

| | |
|---|---|
| N₁₁GCAGCNNNGGATG(WS)ᵢ(WS)ⱼ | (V) |
| N₁₁CGTCGNNNCCTAC(WS)ᵢ | |

where (WS)ᵢ and (WS)ⱼ are each AG, AC, TG, or TC. Mixture (V) is separately ligated to each of mixtures (I)-(IV) to give the four basic reagents for adding dinucleotides to polynucleotides. These tagging reagents can be amplified using a biotinylated LH primer, cut with Bbv 1, and the left hand primer and removed to provide four pools with the structures:

```
5′-p(WS)ᵢ(WS)ⱼAG . . .
                            TC . . .
5′-p(WS)ᵢ(WS)ⱼAC . . .
                            TG . . .
5′-p(WS)ᵢ(WS)ⱼTG . . .
                            AC . . .
5′-p(WS)ᵢ(WS)ⱼTC . . .
                            AC . . .
```

where (WS)ᵢ and (WS)ⱼ are as described above, and p is a phosphate group.

### Arrays of Tag Complements

Complements of ligation tags, referred to herein as “tag complements,” may comprise natural nucleotides or non-natural nucleotide analogs. In one aspect, non-natural nucleic acid analogs are used as tag complements that remain stable under repeated washings and hybridizations of oligonucleoitde tags. In particular, tag complements may comprise peptide nucleic acids (PNAs). Ligation tags from the same minimally cross-hybridizing set when used with their corresponding tag complements provide a means of enhancing specificity of hybridization. Microarrays of tag complements are available commercially, e.g. GenFlex Tag Array (Affymetrix, Santa Clara, Calif.); and their construction and use are disclosed in Fan et al, International patent publication WO 2000/058516; Morris et al, U.S. Pat. No. 6,458,530; Morris et al, U.S. patent publication 2003/0104436; and Huang et al (cited above).

As mentioned above, in one aspect tag complements comprise PNAs, which may be synthesized using methods disclosed in the art, such as Nielsen and Egholm (eds.), Peptide Nucleic Acids: Protocols and Applications (Horizon Scientific Press, Wymondham, UK, 1999); Matysiak et al, Biotechniques, 31: 896-904 (2001); Awasthi et al, Comb. Chem. High Throughput Screen., 5: 253-259 (2002); Nielsen et al, U.S. Pat. No. 5,773,571; Nielsen et al, U.S. Pat. NO. 5,766,855; Nielsen et al, U.S. Pat. No. 5,736,336; Nielsen et al, U.S. Pat. No. 5,714,331; Nielsen et al, U.S. Pat. No. 5,539,082; and the like, which references are incorporated herein by reference. Construction and use of microarrays comprising PNA tag complements are disclosed in Brandt et al, Nucleic Acids Research, 31(19), el 19 (2003).

Preferably, ligation tags and tag complements within a set are selected to have similar duplex or triplex stabilities to one another so that perfectly matched hybrids have similar or substantially identical melting temperatures. This permits mis-matched tag complements to be more readily distinguished from perfectly matched tag complements in the hybridization steps, e.g. by washing under stringent conditions. Guidance for carrying out such selections is provided by published techniques for selecting optimal PCR primers and calculating duplex stabilities, e.g. Rychlik et al, Nucleic Acids Research, 17: 8543-8551 (1989) and 18: 6409-6412 (1990); Breslauer et al, Proc. Nat]. Acad. Sci., 83: 3746-3750 (1986); Wetmur, Crit. Rev. Biochein. Mol. Biol., 26: 227-259 (1991); and the like.

### Hybridization of Labeled Target Sequence to Solid Phase Supports

Methods for hybridizing labeled target sequences to microarrays, and like platforms, suitable for the present invention are well known in the art. Guidance for selecting conditions and materials for applying labeled target sequences to solid phase supports, such as microarrays, may be found in the literature, e.g. Wetmur, Crit. Rev. Biochem. Mol. Biol., 26: 227-259 (1991); DeRisi et al, Science, 278: 680-686 (1997); Chee et al, Science, 274: 610-614 (1996); Duggan et al, Nature Genetics, 21: 10-14 (1999); Schena, Editor, Microarrays: A Practical Approach (IRL Press, Washington, 2000); Freeman et al, Biotechniques, 29: 1042-1055 (2000); and like references. Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference. Hybridization conditions typically include salt concentrations of less than about I M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will stably hybridize to a perfectly complementary target sequence, but will not stably hybridize to sequences that have one or more mismatches. The stringency of hybridization conditions depends on several factors, such as probe sequence, probe length, temperature, salt concentration, concentration of organic solvents, such as formamide, and the like. How such factors are selected is usually a matter of design choice to one of ordinary skill in the art for any particular embodiment. Usually, stringent conditions are selected to be about 5° C. lower than the Tₘ for the specific sequence for particular ionic strength and pH. Exemplary hybridization conditions include salt concentration of at least 0.01 M to no more than 1 M Na ion concentration (or other salts) at a pH 7.0 to 8.3 and a temperature of at least 250 C. Additional exemplary hybridization conditions include the following: 5× SSPE (750 mM NaCl, 50 mM sodium phosphate, 5 mM EDTA, pH 7.4).

Exemplary hybridization procedures for applying labeled target sequence to a GenFlex™ microarray (Affymetrix, Santa Clara, Calif.) is as follows: denatured labeled target sequence at 95-100° C. for 10 minutes and snap cool on ice for 2-5 minutes. The microarray is pre-hybridized with 6× SSPE-T (0.9 M NaCl 60 mM NaH₂,PO₄, 6 mM EDTA (pH 7.4), 0.005% Triton X-100)+0.5 mg/ml of BSA for a few minutes, then hybridized with 120 μL hybridization solution (as described below) at 42° C. for 2 hours on a rotisserie, at 40 RPM. Hybridization Solution consists of 3 M TMACL (Tetramethylammonium. Chloride), 50 mM MES ((2-[N-Morpholino]ethanesulfonic acid) Sodium Salt) (pH 6.7), 0.01 % of Triton X-100, 0.1 mg/ml of Herring Sperm DNA, optionally 50 pM of fluorescein-labeled control oligonucleotide, 0.5 mg/ml of BSA (Sigma) and labeled target sequences in a total reaction volume of about 120 μL. The microarray is rinsed twice with 1× SSPE-T for about 10 seconds at room temperature, then washed with 1× SSPE-T for 15-20 minutes at 40° C. on a rotisserie, at 40 RPM. The microarray is then washed 10 times with 6× SSPE-T at 22° C. on a fluidic station (e.g. model FS400, Affymetrix, Santa Clara, Calif.). Further processing steps may be required depending on the nature of the label(s) employed, e.g. direct or indirect. Microarrays containing labeled target sequences may be scanned on a confocal scanner (such as available commercially from Affymetrix) with a resolution of 60-70 pixels per feature and filters and other settings as appropriate for the labels employed. GeneChip Software (Affymetrix) may be used to convert the image files into digitized files for further data analysis.

### Electrophoretic Readout of Ligation Tags

Ligation tags generated in an analytical process may be identified by grafting them onto members of a set of DNA sequences that may be separated electrophoretically on a conventional DNA sequencing instrument (such DNA sequences are referred to herein as “metric tags”). Briefly, this method of reading out ligation tags provides a one-to-one correspondence between a number of ligation tags in a set and separated DNA sequences in one or more lanes in a DNA sequencing instrument. Thus, for example, say 256 ligation tags were employed in an analytical process that resulted in a subset of the tags that were either labeled or isolated from the rest of the tag set. Also, say that ligation tags I through 256 corresponds to DNA sequences I through 256, which sequences are a nested set of increasing length. If the subset of tags selected consist of tags 47, 62-88, and 195-220, then the selected ligation tags will generate DNA sequences that after separation will occupy bands 47, 62-88, and 195-220. The separated sequences may be labeled directly, or they may be blotted to a solid phase surface and probed with labeled hybridization probes, which may be complements of the ligation tags in some embodiments. The number of DNA sequences per lane is only bounded by the band resolving power of an instrument; thus, the number of DNA sequences per lane may vary from 2 to 1500, or from 2 to 1000. Usually, the number of DNA sequences per lane are in a range of from 50 to 300, or more usually, from 100 to 300. The number of lanes employed is only bound by the practical limitation of commercial electrophoresis instruments and the sorting-by-sequence procedure used to extract DNA sequences for a particular lane. In one aspect, the number of lanes may vary from 1 to 96, reflecting the convenience of working with 96-well plates, or from 1 to 384, or the like. The sorting-by-sequence procedure that is referenced below is disclosed in Appendix I and in pending U.S. patent application Ser. No. 11/055,187, which application is incorporated herein by reference.

In one aspect, the invention is illustrated for the case where there are 256 DNA sequences per lane, and where the sequences are generated from DNAs differing in length by one base and terminated by an appropriate restriction site; each of these are tagged with a tag complement (or ligation anti-tag). In the illustration, four lanes of 256 DNA sequences are described; thus, the illustrated embodiment provides a means of reading out signals for 1024 tags. The following adaptors are employed:

L adaptor (SEQ ID NO: 11):

```
                                                      (b*)
                            Bbv I Bam HI↓
5′-NNNNNNNNNNNGC AGCAAGGATCC
      NNNNNNNNNNNCGTCGTTCCTAGG
```

R adaptor (SEQ ID NO: 12):

```
5′-(G)AGCTCAACCCATCCNNNNNNNNNN-3′
      (C)TCGAGTTGGGTAGGNNNNNNNNNN-5′
            ↑ Sac I Fok I
            (f*)
```

Bbv I has recognition/cleavage properties of 5′-GCAGC(8/12) and Fok I has recognition/cleavage properties 5′-GGATG(9/13), as indicated by the underlining and arrows labeled (b*) and (f*), respectively. The G and C shown in parentheses in the R primer is not part of the adaptor, but will be present to complete the Sac I site. It would be apparent to one of ordinary skill that other adaptors designed for the same purpose using different restriction enzymes would be within the scope of the invention. The Sac I site is used to terminate sequences, the Bam HI site on the L primer is used to interface the anti-coding sequences. In one aspect, a simple repeat sequence, such as [GAAG]ₙ illustrated below, may be used to generate DNA sequences of different lengths for the electrophoresis-based readout. Accordingly, by way of example, the following four oligonucleotides may be synthesized and inserted between the above two adaptors:

| | |
|---|---|
| [L]-GAAGG-[R] | (I) |
| -CTTCC | |
| [L]-GAAGAG-[R] | (II) |
| -CTTCTC | |
| [L]-GAAGAAG-[R] | (ITT) |
| -CTTCTTC | |
| [L]-GAAGGAAG-[R] | (IV) |
| -CTTCCTTC | |

where “[L]” and “[R]” represent the L adaptor and R adaptor described above, respectively. Below, 4 base pairs are added to each to generate inserts of 5, 6, 7, and 8 base pairs. Beginning with the 4 base pair insert, two aliquots are PCR amplified, such that in one aliquot the L primer is biotin labeled, and in the other the R primer is biotin labeled. Cut the L adaptor segment of the amplicon with Bbv I and remove the cleaved adaptor portion with avidinated beads. Likewise, cut the R adaptor segment of the other amplicon with Fok I and remove the cleaved adaptor portion with avidinated beads. These operations leave the following fragments:

In the Bbv I-cleavage reaction:

5′-GAAGGAAG-[R]
CTTC

In the Fok I-cleavage reaction:

[L]-GAAG
-CTTCCTTC-5′

These fragments may be ligated together to generate the following (SEQ ID NO: 13):

[L]-GAAGGAAGGAAG-[R]
-CTTCCTTCCTTC-

which is the 8-nucleotide insert. If a similar operation is carried out using the “L” aliquot of oligonucleotide (III) which give:

5′-GAAGAAG-[R]
TTC-

and ligate it to the “R” aliquot of oligonucleotide (IV) which generates the following:

[L]-GAAG
-CTTCCTTC

then the 7-nucleotide insert is produced. Likewise, oligonucleotides (I) and (II) can be used to generate 5-nucleotide and 6-nucleotide inserts, respectively. If X is the sequence “GAAG,” the remaining DNA sequences may be assembled as follows. Note that (IV) had the capacity to add X and in the same way the 8-nucleotide insert has the capacity to add X-X. Using the 8-nucleotide insert, X-X can be added to 1-nucleotide through 8-nucleotide inserts to generate 9-nucleotide inserts through 16-nucleotide inserts. The 16-nucleotide insert has the structure X-X-X-X-GAAG and it has the capacity to add X-X-X-X, i.e. 16 nucleotides. Using this to add the 16-nucleotides to 1-nucleotide inserts through 16-nucleotide inserts produces 17-nucleotide inserts through 32-nucleotide inserts. In the same way, the remainder of the DNA sequences may be produced so that the total of 256 different-length sequences are obtained.

If it is desired that all of the above DNA sequences be in constructs of the same length, e.g. to facilitate uniform amplification with techniques such as PCR, an analogous system may be implemented to add compensating sequences, e.g. replacing the R primer sites with new R primer sites leaving the Sac I site in the same place.

Ligation anti-tags (or tag complements) are added to the DNA sequences as follows. The ligation codes may be comprised of the following sequences:

5′-WNNZNNW′

where W is G, A, T, or C; N is A, C, G, or T; Z is TG, GT, CA, or AC; and W′ is G when W is G, A when W is A, C when W is T, and T when W is C. An overhang comprising the ligation tag is generated by cleavage with two enzymes as follows (SEQ ID NO: 14):

```
                      N.A1w I ↓ ↓
5′- . . . -GGATCSSSSNNNNNNN . . .
3′ . . . -CCTAGSSSSNNNNNNNSSSSCGCCC . . .
                                                      ↑ Fau I
```

where S and N are separately A, C, G, or T (and complements thereof), and the nucleotides “N” indicate where the overhang occurs after cleavage.

Nucleotides or dinucleotides may be added using Sfa NI. For this purpose, new a new L adaptor is provided with the following design (SEQ ID NO: 15):

5′-N₁₄-GCATCNNNNxTGAA
N₁₄-CGTAGNNNNxACTTCTAp

where N14 is a segment of 14 nucleotides, x=A, and p is a phosphate group. Multiple sets of these 256 adaptors are made. 4 sets are made for x=A, and 4 for all of the others as well in order to make a 4096-member set. Below, a 1024-member set is constructed for x=A.

Cut a sample of each of the 256 DNA sequence tags (i.e. “metric tags” from above) with Bam HI. If, as the last amplification the L primer was labeled with biotin, it can be removed. The cut end is filled in with a G to generate the following ends:

```
                                        Sac I
5′-GATC-[Metric tag]GAGCTC-[R]
            G-[Metric tag]CTCGAG-
```

This is ligated to the starting adaptor to produce (SEQ ID NO: 16):

```
            Sfa NI Sac I
5′-N₁₄GCATCNNNNATGAAGATCC[Metric tag]GAGGTC-[R]
      N₁₄CGTAGNNNNTACTTCTAGG[Metric tag]CTCGAG-
                                        N.A1w I
```

Doublets, or dinucleotides, are added to the first 16 metric tags using previous techniques. Note the correspondence of the doubletto the number (or length) of the tag. This is done four times using tags 1-64 and pool the batches of 16, to each of these are added doublets TG, GT, CA, and AC, and then pool, noting again the correspondence. This is done with tags 65 to 128, 129-192 and 193-256, and to each of these add a single base, and pool. This allocates all of the tags. Four samples of these pools are taken and to each a new left hand adaptor shown below is added (SEQ ID NO: 17):

5′-N₁₅CCCGCNNNN(A*)z
Fau I

where z is A, G, C, or T, and (A*) is determined by how the process is started. This completes the set for 1024 with 4 groups of nucleotides. The 4 sets are mixed. For 4096, the process is repeated four times using a different nucleotide for the outer states. These 16 sets can be pooled together. Note that besides Sfa NI used above, any enzyme which does not cut the ligation codes may be used, such as Btg ZI which cuts at GCGATG(10/14) or Fau I which cuts at CCCGC(4/6).

After sorting by sequence, all the templates and their accompanying tags are sorted into separate compartments according to the base at that position. The ligation codes lay between two adaptors R₁ and R₂ and, in the case of double tagging, there is an additional site between R₂ and R₃. An enzyme, such as Eco NI, which does not cut the ligation codes is used (SEQ ID NO: 18):

```
                ↓
5′-CCTNNNNNAGG-
    -GGANNNNNTCC-
                  ↑
```

The original R₁ has the structure containing the nicking enzyme (SEQ ID NO: 19):

5′-N₁₆CCTAGTCTAGGN₇GGATCNNNN-[Ligation codes]
N₁₆GGATCAGATCCN₇CCTAGNNNN-

Single stranded DNAs of the correct polarity are generated by the sequence by sorting method so that they may be used directly after release in the next step. An RI primer of the following structure is used (SEQ ID NO: 20):

5′-₁₆CCTAGxCTAGGN₇GGATC

where x=T for the A-compartment and x=A, G and C for the T-, C—, and G-compartments, respectively. This primer is biotinylated, allowing the copies made to be removed. These in turn can be copied and then amplified using the R₁* primer: N₁₆CCTAG and a primer for the R₂ (or R₃) adaptor, which can be labeled with biotin. After cutting with the nicking enzyme and Fau I to reveal the single stranded ligation codes, the right hand fragments are removed. The collection of metric tags with the left hand adaptor labeled with biotin at the last PCR is similarly cut to reveal the complementary single stranded anti-ligation tags, and the two are hybridized together and ligated.

Once the metric tags are attached, processing proceeds as follows. Cut with Eco NI to fragment the tags into two pieces (SEQ ID NO: 21):

| | |
|---|---|
| 5′-N₁₆CCTAG | xCTAGGN₇GGATCNNNN . . . |
| and | |
| N₁₆GGATCx | GATCCN₇CCTAGNNNN . . . |

(The left hand fragments may be removed using another ligand system, such as methotrexate, although it is not absolutely necessary and a mixture of dideoxynucleotide terminators may be used to label both fragments, but the second is selected in the next step). Cut with Sac I to terminate the metric tags, to give from the following (SEQ ID NO: 22):

```
5′-xCTAGGN₇GGATCNNNN[LIG-8][GGAG]BₙGAGTCT . . .
      xGATCCN₇CCTAGNNNN[LIG-8][GGAG]BₙCTCAGA . . .
                                                                      Sac I
```

where n ranges from 0 to 255, the fragments (SEQ ID NO: 23):

5′-xCTAGGN₇GGATCNNNN[LIG-8][GGAG]BₙGAGTC
xGATCCN₇CCTAGNNNN[LIG-8][GGAG]BₙC

whose top strands are digested with T7 exonuclease, or like enzyme that does not cut recessed 5′ ends. This will also remove the left hand fragments or at least reduce their molecular weight.

The final step is to sort the lower strands into different sets. The following primer common to all the strands is employed (SEQ ID NO: 24):

CTAGGN₇GGATCN₄

The first base is sorted for, then using 4 primers with A, G, C, or T, the second set is sorted for, to give the 16 sets for 4096. If only 1024 is being used, as in the example indicated above where the first base is known to be A, then only that primer need be used and only 4 channels need be run. For example, on a 96-channel Applied Biosystems DNA sequencer, 24 sets of 4 can be run in one run.

### Translating Binary Tags Into Metric Taos For An Electrophoretic Readout

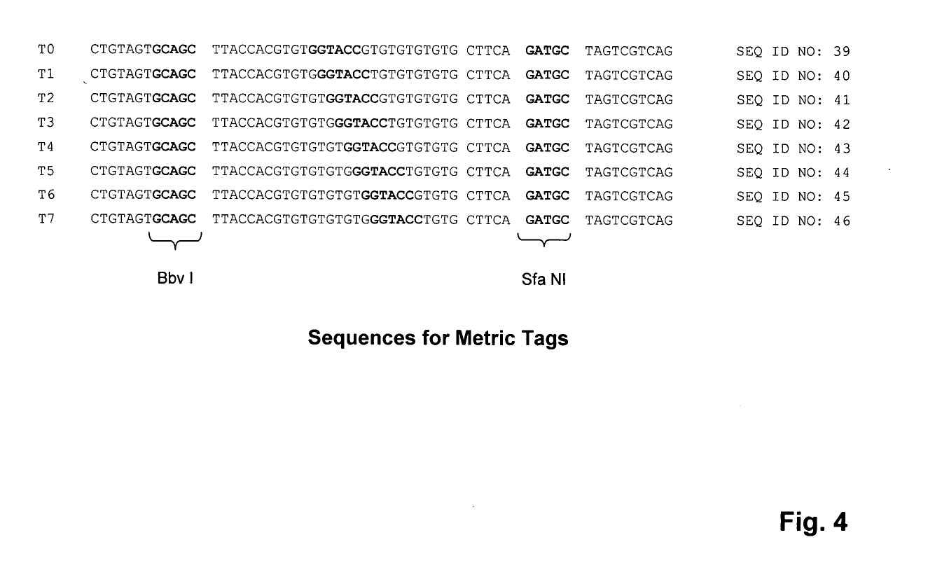

In this example, binary tags of 512 fragments are recoded as metric tags that can be readout by electrophoretic separation. The following reagents are synthesized using conventional methods:

| | | |
|---|---|---|
| | Bbv I Sfa NI | |
| | ↓ ↓ | |
| S₀ | N₇GCAGCN₈(TG)₆N₅GATGCN₁₀ | (SEQ ID NO: 25) |
| | N₇CGTCGN₈(AC)₆N₅CTACGN₁₀ | |
| | RH | |
| | [symbol] | |
| | Bbv I Sfa NI | |
| | ↓ ↓ | |
| T₀ | N₇GCAGCN₈TGTGGTACCGTGTGTGTGTGN₅GATGCN₁₀ | (SEQ ID NO: 26) |
| | N₇CGTCGN₈ACACCATGGCACACACACACN₅CTACGN₁₀ | |
| T₁ | N₇GCAGCN₈TGTGGGTACCTCTCTGTGTGN₅GATGCN₁₀ | (SEQ ID NO: 27) |
| | N₇CGTCGN₈ACACCCATGGACACACACACN₅CTACGN₁₀ | |
| T₂ | N₇GCAGCN₈TGTGTGGTACCGTGTGTGTGN₅GATGCN₁₀ | (SEQ ID NO: 28) |
| | N₇CGTCGN₈ACACACCATGGCACACACACN₅CTACGN₁₀ | |
| T₃ | N₇GCAGCN₈TGTGTGGGTACCTGTCTGTGN₅GATGCN₁₀ | (SEQ ID NO: 29) |
| | N₇CGTCGN₈ACACACCCATGGACACACACN₅CTACGN₁₀ | |
| T₄ | N₇CCAGCN₈TGTGTGTGGTACCGTCTCTCN₅GATGCN₁₀ | (SEQ ID NO: 30) |
| | N₇CGTCGN₈ACACACACCATGGCACACACN₅CTACGN₁₀ | |
| T₅ | N₇GCAGCN₈TGTGTGTGGGTACCTGTGTGN₅GATGCN₁₀ | (SEQ ID NO: 31) |
| | N₇CGTCGN₈ACACACACCCATGGACACACN₅CTACGN₁₀ | |
| T₆ | N₇GCAGCN₈TGTGTGTGTGGTACCGTGTGN₅GATGCN₁₀ | (SEQ ID NO: 32) |
| | N₇CGTCGN₈ACACACACACCATGGCACACN₅CTACGN₁₀ | |
| T₇ | N₇GCAGCN₈TGTGTGTGTCGGTACCTGTGN₅GATGCN₁₀ | (SEQ ID NO: 33) |
| | N₇CGTCGN₈ACACACACACCCATGGACACN₅CTACGN₁₀ | |

where the bolded letters indicate the position of a Kpn I site. The upper stands of the above sequences are also shown in the table of FIG. 4 with exemplary express sequences inserted for the N's shown above. From these components, Sₒ can be concatenated to give different lengths of insert in multiples of eight bases in accordance with the formula: Sᵢ=nSₒ with biotinylated left hand primer and separately with biotinylated right hand primer. The above are processed by cutting with Bbv I and removing the left end to leave (SEQ ID NO: 34):

| | |
|---|---|
| RH end | |
| [symbol] | |
| TGTGTGTGN₅GATGCN₁₀ | (A) |
| pACACACACACACN₅CTACGN₁₀ | |

Separately cut RH end with Sfa NI and remove the right end to leave (SEQ ID NO: 35):

| | | |
|---|---|---|
| LH end | TGTGTGTGTGTGp | (B) |
| | ACACACAC | |

(A) and (B) are ligated and amplified by PCR to provide a reagent, S₂, for adding 16 bases. S₃ is made by the same method from S₁ and S₂, and S₄ from S, and S₂. Likewise, S₅ through S₈ are constructed by similar combinations as follows.

| | | Bases Added By |
|---|---|---|
| Concatenate | Resulting Reagent | Concatenate |
| S1 + S2 | S3 | 24 |
| S2 + S2 | S4 | 32 |
| S1 + S4 | S5 | 40 |
| S2 + S4 | S6 | 48 |
| S3 + S4 | S7 | 56 |
| S4 + S4 | S8 | 64 |

Call the last reagent a “block” or S8=B1. Using the same methods, B2 to B7 are constructed for adding bases in multiples of 64.

Recall that the final tagged library has the following structure (SEQ ID NO: 36):

| | | |
|---|---|---|
| L | R1 | R2 |
| [symbol] | [symbol] | [symbol] |
| N₁₄TCCAACN₁₈(WS)ᵢ | N₆[insert] | N₂₀ TGTGN₅GATGCN₁₀ |
| N₁₄AGGTTGN₁₈(WS)ᵢ | N₆[insert] | N₂₀ ACACN₅CTACGN₁₀ |
| \| | | |

Mme I site
   where (WS)i is AG, AC, TG, or TC. The ends of this structure is modified as follows. This left end is designed for addition of dinucleotide units. This design is changed so that dinucleotide units can be removed. The objective is to produce an element with the form (SEQ ID NO: 37):
   N₁₄N₃(WS)ᵢN₂ . . .
   N₁₄N₃(WS)ᵢN₂ . . .
   It could be substituted now or it could be used in the last tagging set of adaptors.

Single strands for sorting are obtained and at the same time the methylated Sfa NI site on the right is unblocked. Using an R2 primer the denatured DNA is copied once to displace the old bottom strand, which is destroyed by addition of exonuclease I. After heat deactivation of the enzyme, more primer is added and the amplification is repeated several times, e.g. 8 times. The sorting proceeds by alternative extension with dGTP or dCTP and with dTTP or dATP. The resulting strands are hybridized to a biotinylated L primer and moved to a new solution. All these are one-tube reactions. The top strand is now primed with R1 and extended to make the right end double stranded. Strands can now be sorted from the left end. Using the dideoxy method, successively synthesized primers are used to perform the first sort. Thus, if the first sort is G v C, then two primers, one extended by G and the other by C are required for the sort. The next step, sorting again for G v C, requires four primers, the original, pₒ, extended by GA, GT, CA, CT. Any further sorting would require the synthesis of additional primers. In the case considered here, the binary code is used twice, and so the alternative, remove 3 bases and start again, cannot be used. Here it is essential to use the process of detaching the ligand, so that the primer is extended at the same time as sorting. Another possibility is to synthesize the primer in steps, after separation and release.

Recoding is implemented as follows. Remove the right end of the above by cutting with Sfa NI. Sort into eight batches. A binary number can be assigned to these, on the convention that A=0, T=1, and G=0, C=1 (i.e. R=0, Y=1). In ascending numerical order, ligate as follows: 000, no addition, 001 B1 (that is, 1 block 64 bases), 010 B2, and so on up to 111, B7 pool, cut right end and sort into next 8 classes. Using same numbering rule, add to 000 nothing, to 001, S1, which adds 8 bases, to 010, S2 to add 16 bases and so on until 111 receives S7, which adds 56 bases. Again, after ligation, pool and cut. Now again sort a further 3 steps into eight batches. Again, these are labeled 000 to 111, and now these are added to as follows: 000, TO, 001, T1, and so on until 111 receives T7. Sequences have now been added that will give eight separate bands upon electrophoretic separation, stepped by one nucleotide, when the tags are processed. The process is completed as follows. Although each genome is in a one-to-one correspondence with a single length of an oligonucleotide (i.e. a metric tag), the physical lengths of the metric tags are not the same and since it is desirable to be able to PCR the tags, preferably the metric tags should be the same length. Thus, appropriate length of oligonucleotide are added to each to make them all the same. Remove the primers, make all of the DNA double stranded (amplify if necessary), make it single stranded at the left end (as before), and double stranded at the right. Sort into 8 batches for block addition, number from 000 to 111. Add blocks but in reverse order: to 000 add B7, 001 B6 and so on until 111 receives nothing. Pool, cut again at right end, sort into 8 batches, number from 000 to 111 and add Sn, n=1, 2 . . . 7, in reverse order, such that 000 receives S7, 001 S6, and so on until 111 receives nothing. Pool again, cut and add an appropriate final end required for subsequent steps. Note although there is not a symmetrical disposition of blocks and steps, we have BS-sequence-BS, it does not matter because now every tag now has the same length.

The above teachings are intended to illustrate the invention and do not by their details limit the scope of the claims of the invention. While preferred illustrative embodiments of the present invention are described, it will be apparent to one skilled in the art that various changes and modifications may be made therein without departing from the invention, and it is intended in the appended claims to cover all such changes and modifications that fall within the true spirit and scope of the invention.

### Appendix I

### Sequence-Specific Sorting

Sequence-specific sorting, or sorting by sequence, is a method for sorting polynucleotides from a population based on predetermined sequence characteristics, as disclosed in Brenner, PCT publication WO 2005/080604 and below. In one aspect, the method is carried out by the following steps: (i) extending a primer annealed polynucleotides having predetermined sequence characteristics to incorporate a predetermined terminator having a capture moiety, (ii) capturing polynucleotides having extended primers by a capture agent that specifically binds to the capture moiety, and (iii) melting the captured polynucleotides from the extended primers to form a subpopulation of polynucleotides having the predetermined sequence characteristics.

The method includes sorting polynucleotides based on predetermined sequence characteristics to form subpopulations of-reduced complexity. In one aspect, such sorting methods are used to analyze populations of uniquely tagged polynucleotides, such as genome fragments. During or at the conclusion of repeated steps of sorting in accordance with the invention, the tags may be replicated, labeled and hybridized to a solid phase support, such as a microarray, to provide a simultaneous readout of sequence information from the polynucleotides. As described more fully below, predetermined sequence characteristics include, but are not limited to, a unique sequence region at a particular locus, a series of single nucleotide polymorphisms (SNPs) at a series of loci, or the like. In one aspect, such sorting of uniquely tagged polynucleotides allows massively parallel operations, such as simultaneously sequencing, genotyping, or haplotyping many thousands of genomic DNA fragments from different genomes.

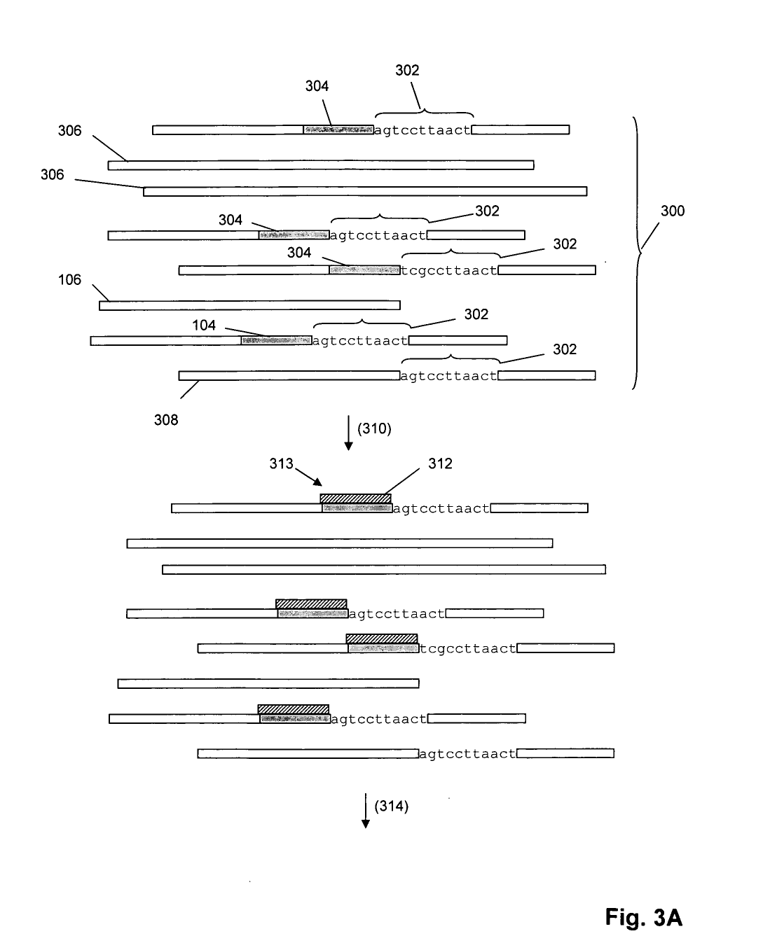

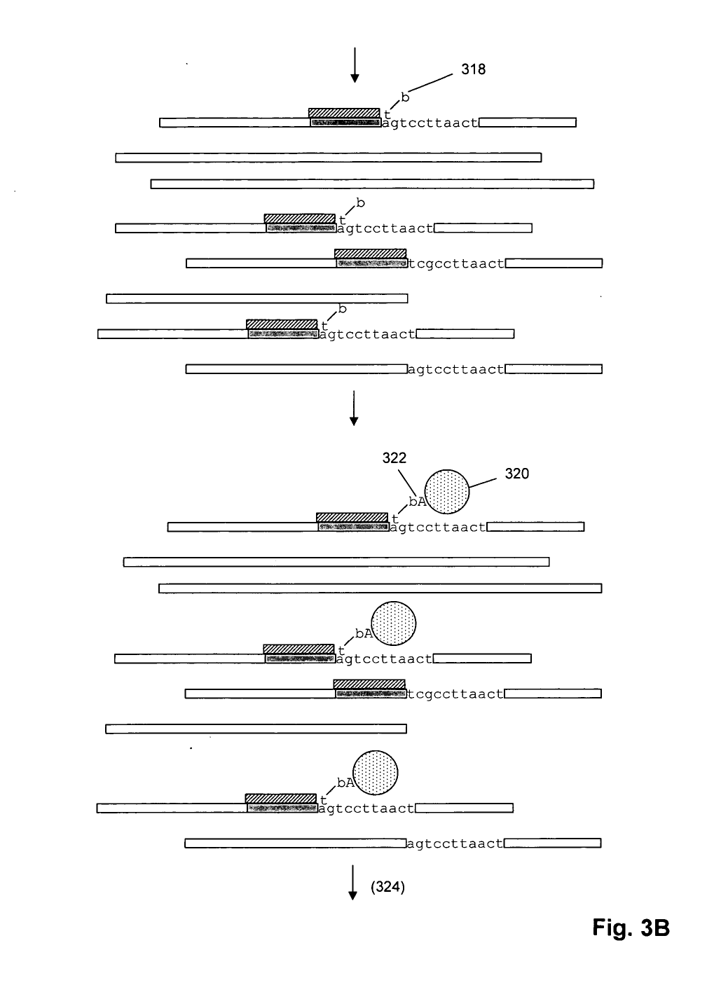

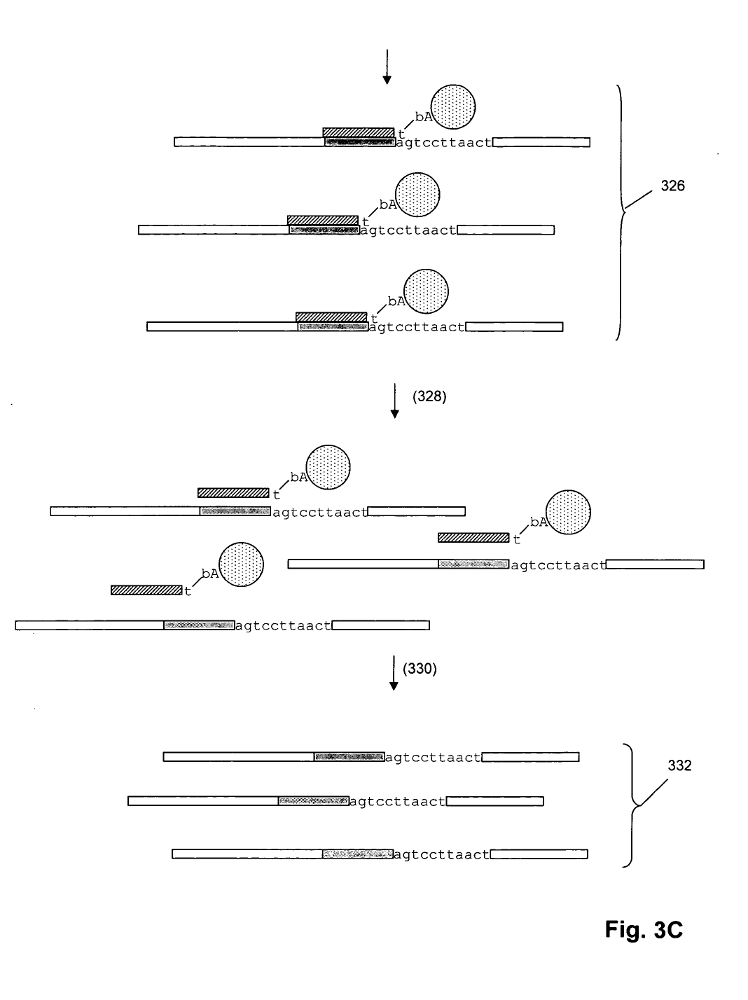

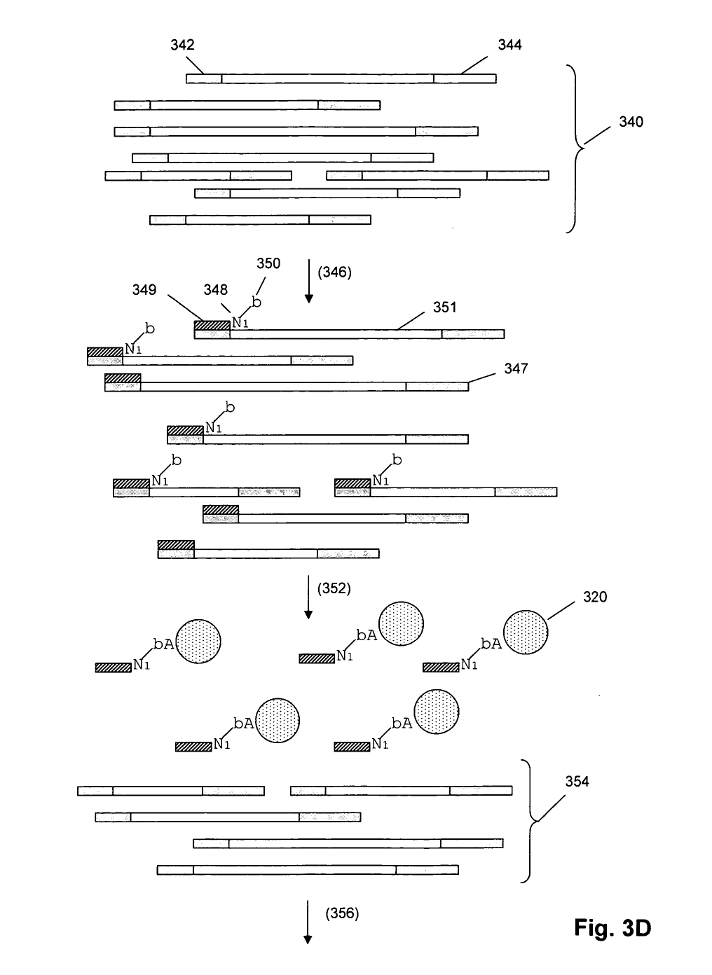

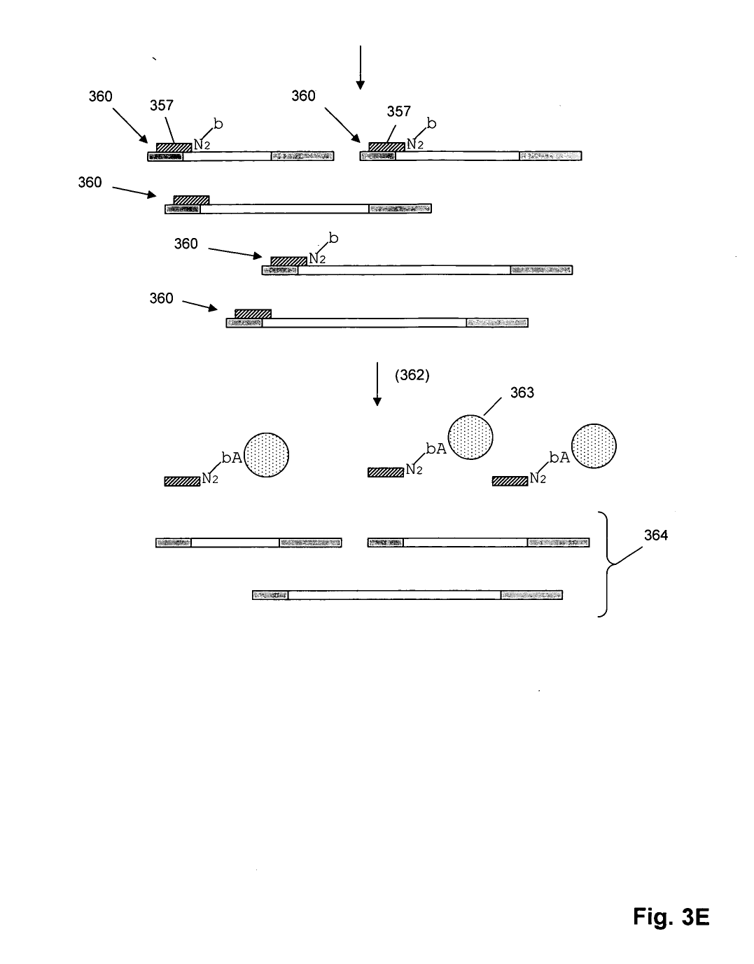

One aspect of the complexity-reducing method of the invention is illustrated in FIGS. 3A-3C. Population of polynucleotides (300), sometimes referred to herein as a parent population, includes sequences having a known sequence region that may be used as a primer binding site (304) that is immediately adjacent to (and upstream of) a region (302) that may contain one or more SNPs. Primer binding site (304) has the same, or substantially the same, sequence whenever it is present. That is, there may be differences in the sequences among the primer binding sites (304) in a population, but the primer selected for the site must anneal and be extended by the extension method employed, e.g. DNA polymerase extension. Primer binding site (304) is an example of a predetermined sequence characteristic of polynucleotides in population (300). Parent population (300) also contains polynucleotides that do not contain either a primer binding site (304) or polymorphic region (302). In one aspect, the invention provides a method for isolating sequences from population (300) that have primer binding sites (304) and polymorphic regions (302). This is accomplished by annealing (310) primers (312) to polynucleotides having primer binding sites (304) to form primer-polynucleotide duplexes (313). After primers (312) are annealed, they are extended to incorporate a predetermined terminator having a capture moiety. Extension may be effected by polymerase activity, chemical or enzymatic ligation, or combinations of both. A terminator is incorporated so that successive incorporations (or at least uncontrolled successive incorporations) are prevented.

This step of extension may also be referred to as “template-dependent extension” to mean a process of extending a primer on a template nucleic acid that produces an extension product, i.e. an oligonucleotide that comprises the primer plus one or more nucleotides, that is complementary to the template nucleic acid. As noted above, template-dependent extension may be carried out several ways, including chemical ligation, enzymatic ligation, enzymatic polymerization, or the like. Enzymatic extensions are preferred because the requirement for enzymatic recognition increases the specificity of the reaction. In one aspect, such extension is carried out using a polymerase in conventional reaction, wherein a DNA polymerase extends primer (312) in the presence of at least one terminator labeled with a capture moiety. Depending on the embodiment, there may be from one to four terminators (so that synthesis is terminated at any one or at all or at any subset of the four natural nucleotides). For example, if only a single capture moiety is employed, e.g. biotin, extension may take place in four separate reactions, wherein each reaction has a different terminator, e.g. biotinylated dideoxyadenosine triphosphate, biotinylated dideoxycytidine triphosphate, and so on. On the other hand, if four different capture moieties are employed, then four terminators may be used in a single reaction. Preferably, the terminators are dideoxynucleoside triphosphates. Such terminators are available with several different capture moieties, e.g. biotin, fluorescein, dinitrophenol, digoxigenin, and the like (Perkin Elmer Lifesciences). Preferably, the terminators employed are biotinylated dideoxynucleoside triphosphates (biotin-ddNTPs), whose use in sequencing reactions is described by Ju et al, U.S. Pat. No. 5,876,936, which is incorporated by reference. In one aspect of the invention, four separate reactions are carried out, each reaction employing only one of the four terminators, biotin-ddATP, biotin-ddCTP, biotin-ddGTP, or biotin-ddTTP. In further preference, in such reactions, the ddNTPs without capture moieties are also included to minimize mis-incorporation. As illustrated in FIG. 3B, primer (312) is extended to incorporate a biotinylated dideoxythymidine (318), after which primer-polynucleotide duplexes having the incorporated biotins are captured with a capture agent, which in this illustration is an avidinated (322) (or streptavidinated) solid support, such as a microbead (320). Captured polynucleotides (326) are separated (328) and polynucleotides are melted from the extended primers to form (330) population (332) that has a lower complexity than that of the parent population (300). Other capture agents include antibodies, especially monoclonal antibodies that form specific and strong complexes with capture moieties. Many such antibodies are commercially available that specifically bind to biotin, fluorescein, dinitrophenol, digoxigenin, rhodamine, and the like (e.g. Molecular Probes, Eugene, Oreg.).

The method also provides a method of carrying out successive selections using a set of overlapping primers of predetermined sequences to isolate a subset of polynucleotides having a common sequence, i.e. a predetermined sequence characteristic. By way of example, population (340) of FIG. 3D is formed by digesting a genome or large DNA fragment with one or more restriction endonucleases followed by the ligation of adaptors (342) and (344), e.g. as may be carried out in a conventional AFLP reactions, U.S. Pat. No. 6,045,994, which is incorporated herein by reference. Primers (349) are annealed (346) to polynucleotides (351) and extended, for example, by a DNA polymerase to incorporate biotinylated (350) dideoxynucleotide N. (348). After capture (352) with streptavidinated microbeads (320), selected polynucleotides are separated from primer-polynucleotide duplexes that were not extended (e.g. primer-polynucleotide duplex (347)) and melted to give population (354). Second primers (357) are selected so that when they anneal they basepair with the first nucleotide of the template polynucleotide. That is, their sequence is selected so that they anneal to a binding site that is shifted (360) one base into the polynucleotide, or one base downstream, relative to the binding site of the previous primer. That is, in one embodiment, the three-prime most nucleotide of second primers (357) is N₁. In accordance with the invention, primers may be selected that have binding sites that are shifted downstream by more than one base, e.g. two bases. Second primers (357) are extended with a second terminator (358) and are captured by microbeads (363) having an appropriate capture agent to give selected population (364). Successive cycles of annealing primers, extension, capture, and melting may be carried out with a set of primers that permits the isolation of a subpopulation of polynucleotides that all have the same sequence at a region adjacent to a predetermined restriction site. Preferably, after each cycle the selected polynucleotides are amplified to increase the quantity of material for subsequent reactions. In one aspect, amplification is carried out by a conventional linear amplification reaction using a primer that binds to one of the flanking adaptors and a high fidelity DNA polymerase. The number of amplification cycles may be in the range of from I to 10, and more preferably, in the range of from 4 to 8. Preferably, the same number of amplification cycles is carried out in each cycle of extension, capturing, and melting.

### Advancing Along a Template by “Outer Cycles” of Stepwise Cleavage

The above selection methods may be used in conjunction with additional methods for advancing the selection process along a template, which allows sequencing and/or the analysis of longer sections of template sequence. A method for advancing a template makes use of type I Is restriction endonucleases, e.g. Sfa NI (5′-GCATC(5/9)), and is similar to the process of “double stepping” disclosed in U.S. Pat. No. 5,599,675, which is incorporated herein by reference. “Outer cycle” refers to the use of a type IIs restriction enzyme to shorten a template (or population of templates) in order to provide multiple starting points for sequence-based selection, as described above. In one aspect, the above selection methods may be used to isolate fragments from the same locus of multiple genomes, after which multiple outer cycle steps, e.g. K steps, are implemented to generated K templates, each one successively shorter (by the “step” size, e.g. 1-20 nucleotides) than the one generated in a previous iteration of the outer cycle. Preferably, each of these successively shortened templates is in a separate reaction mixture, so that “inner” cycles of primer extensions and sortings can be implemented of the shortened templates separately.

In another aspect, an outer cycle is implemented on a mixture of fragments from multiple loci of each of multiple genomes. In this aspect, the primer employed in the extension reaction (i.e. the inner cycle) contains nucleotides at its 3′ end that anneal specifically to a particular locus, and primers for each locus are added successively and a selection is made prior to the next addition of primers for the next locus.

Assume that starting material has the following form (SEQ ID NO: 45) (where the biotin is optional):

biotin-NN . . . NNGCATCAAAAGATCNN . . .
NN . . . NNCGTAGTTTTCTAGNN . . .

and that after cleavage with Sfa NI the following two fragments are formed (SEQ ID NO: 46):

| | |
|---|---|
| biotin-NN . . . NNGCATCAAAAG | pATCNN . . . |
| NN . . . NNCGTAGTTTTCTAGNp | N . . . |

where “p” designates a 5′ phosphate group. The biotinylated fragments are conveniently removed using conventional techniques. The remaining fragments are treated with a DNA polymerase in the presence of all four dideoxynucleoside triphosphates to create end on the lower strand that cannot be ligated:
pATCN NN . . .
N_{dd}NN . . .

where “N_{dd}” represents an added dideoxynucleotide. To these ends are ligated adaptors of the following form (SEQ ID NO: 47):

N*N*N*NN . . . NNNGCATCAAAA
N N N NN . . . NNNCGTAGTTTTNNN

where “N*” represents a nucleotide having a nuclease-resistant linkage, e.g. a phosphorothioate. The specificity of the ligation reaction is not crucial; it is important merely to link the “top” strands together, preserving sequence. After ligation the following structure is obtained (SEQ ID NO: 48):

N*N*N*NN . . . NNNGCATCAAAAATCN N . . .
N N N NN . . . NNNCGTAGTTTTNNNN_{dd}N . . .

The bottom strand is then destroyed by digesting with T7 exonuclease 6, λ exonuclease, or like enzyme. An aliquot of the remaining strand may then be amplified using a first primer of the form:

5′-biotin-NN . . . GCATCAAAA

and a second primer containing a T7 polymerase recognition site. This material can be used to re-enter the outer cycle. Another aliquot is amplified with a non-biotinylated primer (5′-NN . . .

GCATCAAAA) and a primer containing a T7 polymerase recognition site eventually to produce an excess of single strands, using conventional methods. These strands may be sorted using the above sequence-specific sorting method where “N” (italicized) above is G, A, T, or C in four separate tubes.

The basic outer cycle process may be modified in many details as would be clear to one of ordinary skill in the art. For example, the number of nucleotides removed in an outer cycle may vary widely by selection of different cleaving enzymes and/or by positioning their recognition sites differently in the adaptors. In one aspect, the number of nucleotides removed in one cycle of an outer cycle process is in the range of from 1 to 20; or in another aspect, in the range of from 1 to 12; or in another aspect, in the range of from 1 to 4; or in another aspect, only a single nucleotide is removed in each outer cycle. Likewise, the number of outer cycles carried out in an analysis may vary widely depending on the length or lengths of nucleic acid segments that are examined. In one aspect, the number of cycles carried out is in the range sufficient for analyzing from 10 to 500 nucleotides, or from 10 to 100 nucleotides, or from 10 to 50 nucleotides.

In one aspect of the invention, templates that differ from one or more reference sequences, or haplotypes, are sorted so that they may be more fully analyzed by other sequencing methods, e.g. conventional Sanger sequencing. For example, such reference sequences may correspond to common haplotypes of a locus or loci being examined. By use of outer cycles, actual reagents, e.g. primers, having sequences corresponding to reference sequences need not be generated. If at each extension (or inner) cycle, either each added nucleotide has a different capture moiety, or the nucleotides are added in separate reaction vessels for each different nucleotide. In either case, extensions corresponding to the reference sequences and variants are immediately known simply by selecting the appropriate reaction vessel or capture agents.

What is claimed is:

1. A method of identifying a segmented tag by size separation, the method comprising the steps of:
providing a segmented tag comprising more than one subunits, each subunit having a position in the segmented tag and each being selected from a set of subunits consisting of a plurality of different nucleotides or oligonucleotides;
providing for each position of the segmented tag a fragment set, such fragment sets having successively larger nucleic acid fragments such that a shortest nucleic acid fragment of a next-larger fragment set has a length that is greater than or equal to that of a longest nucleic acid fragment of a next-smaller fragment set, and wherein each nucleic acid fragment within a fragment set has a different length and each fragment within a set has a one-to-one correspondence with a different subunit;
concatenating for each position of the segmented tag a nucleic acid fragment from its corresponding fragment set, each such nucleic acid fragment corresponding to the subunit at the position corresponding to its fragment set to form a concatenate; and
determining the length of the concatentate to identify the segmented tag.

2. The method of claim 2 wherein said segmented tag is a sequence of nucleotides.

3. The method of claim 1 wherein said segmented tag comprises a sequence of oligonucleotide subunits each having a length in the range of from 2 to 12 nucleotides.

4. The method of claim 3 wherein said segmented tag is a sequence of dinucleotide tags.

5. The method of claim 3 wherein said segmented tag is a ligation tag.

6. A method of identifying members of a population of segmented tags, wherein each segmented tag of the population comprises a sequence of subunits selected from a plurality of different nucleotides or oligonucleotides, each subunit having a position within a segmented tag, the method comprising the steps of:
(a) providing for each position of the segmented tags a fragment set, such fragment sets having successively larger nucleic acid fragments such that a shortest nucleic acid fragment of a next-larger fragment set has a length that is greater than or equal to that of a longest nucleic acid fragment of a next-smaller fragment set, and wherein each nucleic acid fragment within a fragment set has a different length and each fragment within a set has a one-to-one correspondence with a different subunit;
(b) concatenating for each position of each segmented tag nucleic acid fragments from the fragment set corresponding to each such position and corresponding to the subunit occupying such position to form for each segmented tag a concatenate; and
(c) separating the concatenates by length to identify the corresponding segmented tags.

7. The method of claim 6 wherein said step of concatenating includes:
(i) sorting said segmented tags into a plurality of groups according to the identity of a subunit at a position within said segmented tags, said segmented tags having not been sorted previously from such position;
(ii) attaching to each segmented tag of each group a fragment corresponding to the subunit of such group to form concatenates;
(iii) combining the concatenates; and
(iv) repeating steps (i) through (iii) until the segmented tags have been sorted at each position.

8. The method of claim 7 wherein each of said segmented tags is a sequence of nucleotides.

9. The method of claim 7 wherein each of said segmented tags comprises a sequence of oligonucleotide subunits each having a length in the range of from 2 to 12 nucleotides.

10. The method of claim 3 wherein each of said segmented tags is a sequence of dinucleotide tags.

11. The method of claim 3 wherein each of said segmented tags is a ligation tag.

12. A set of ligation tags comprising a plurality of member oligonucleotides, each such member having a tag complement and each comprising:
a length in the range of from six to twelve nucleotides;
a duplex stability with its tag complement equivalent to that of every other oligonucleotide member;
a first terminal nucleotide and a second terminal nucleotide selected so that whenever a member oligonucleotide forms a duplex with a tag complement of another member oligonucleotide, the first terminal nucleotide and the second nucleotide each form mismatches with respect to nucleotides of the tag complement with which they are paired.

13. A method of identifying individual polynucleotides in a mixture, the method comprising the steps of:
attaching to each individual polynucleotide in the mixture a different ligation tag to form tag-polynucleotide conjugates;
generating labeled ligation tags from the tag-polynucleotide conjugates; and
identifying the labeled ligation tags on a readout platform.

14. The method of claim 13 wherein said readout platform is a microarray.

15. The method of claim 13 wherein said readout platform is a DNA separation instrument and wherein said step of generating further includes the steps of attaching a metric tag to each of said tag-polynucleotide conjugates to form a metric tag-ligation tag conjugate, such that each of said ligation tags is conjugated to a unique metric tag; and separating and detecting the metric tag-ligation conjugates with the DNA separation instrument.

16. A method of generating a single stranded overhang in a cleavage of a double stranded DNA, the method comprising the steps of:
providing a first recognition site of a nicking enzyme in a double stranded DNA, the nicking enzyme being capable of cleaving only a single strand of the double stranded DNA;
providing a second recognition site of a restriction endonuclease in the double stranded DNA, the restriction endonuclease being capable of cleaving both strands of the double stranded DNA,
providing a cleavage segment in the double stranded DNA, the cleavage segment being disposed between and being immediately adjacent to the first recognition site and the second recognition site; and
cleaving the double stranded DNA with the nicking enzyme and the restriction endonuclease so that at a first end of the cleavage segment both strands of the double stranded DNA are cleaved and at a second end of the cleavage segment a single strand of the double stranded DNA is cleaved to produce a free cleavage segment oligonucleotide and a single stranded overhang.

17. The method of claim 5 wherein said cleavage segment has a nucleotide sequence, wherein said nicking enzyme is a type IIs nicking enzyme having a cleavage site separate from said first recognition site, and wherein said restriction endonuclease is a type IIs restriction endonuclease having a cleavage site separate from said second recognition site, so that the nucleotide sequence of said cleavage segment is independent of either said first or second recognition sites.

18. A composition of matter comprising a plurality of ligation tags selected from the group defined by the formulas:
5′-Y₁—N₁N₂-(Z)_{K}-N₃N₄—Y₂
where K is 1, 2, or 3; Y. and Y₂ are separately each A, C, G, or T; N₁, N₂, N₃, and N₄ are separately each A, C, G, or T; and Z is a dinucleotide, GT, TG, CA, or AC, with the proviso that whenever K is greater than one, each Z is separately GT, TG, CA, or AC.

19. The composition of claim 18 wherein said plurality is at least 100 and wherein Y₂ is T whenever Y₁ is G, and Y₂ is C whenever Y₁ is A, and Y₂ is G whenever Y₁ is T, and Y₂ is A whenever Y₁ is C.

20. The composition of claim 19 wherein said ligation tags contain no dinucleotides having a sequence CC, GC, GG, or CG and every ligation tag of said plurality has a sequence that differs from that of every other ligation tag of the same plurality by at least two nucleotides.

21. The composition of claim 20 wherein K is 1 or 2.
